(19) United States
(12) Patent Application Publication
(10) Pub. No.: US 2025/0057994 A1
(43) Pub. Date: Feb. 20, 2025

(54) LABELED COMPOUNDS AND CALCIUM SENSING RECEPTOR LIGANDS FOR IMAGING AND USES THEREOF

(71) Applicant: The University of North Carolina at Chapel Hill, Chapel Hill, NC (US)

(72) Inventors: Zibo Li, Chapel Hill, NC (US); Lawrence Kim, Chapel Hill, NC (US); Zhanhong Wu, Chapel Hill, NC (US)

(21) Appl. No.: 18/578,434

(22) PCT Filed: Jul. 11, 2022
(86) PCT No.: PCT/US2022/036645

§ 371 Date: Jan. 11, 2024

Related U.S. Application Data
(60) Provisional application No. 63/220,737, filed on Jul. 12, 2021.

(51) Int. Cl. A61K 51/04

(52) U.S. Cl. CPC A61K 51/0446; A61K 51/0402; A61K 2123/00

(57) ABSTRACT

The present invention relates to labeled compounds suitable for positron emission tomography (PET) imaging and/or fluorescence imaging such as near-infrared fluorescence imaging (NIRF). The invention further relates to the use of these compounds for carrying out PET scans. imaging calcium sensing receptor (CSR)-positive organs. excising parathyroid tissue. protecting parathyroid tissue during thyroid surgery, and treating disorder(s) of a CSR-positive tissue in a subject.

### STATEMENT OF PRIORITY

This application claims the benefit of U.S. Provisional Application Ser. No. 63/220,737, filed Jul. 12, 2021, the entire contents of which are incorporated by reference herein.

### STATEMENT OF GOVERNMENT SUPPORT

This invention was made with government support under Grant No. DK128447 awarded by the National Institutes of Health. The government has certain rights in the invention.

### FIELD OF THE INVENTION

The present invention relates to labeled compounds suitable for positron emission tomography (PET) imaging and/or fluorescence imaging such as near-infrared fluorescence imaging (NIRF). The invention further relates to the use of these compounds for carrying out PET scans, imaging calcium sensing receptor (CSR)-positive organs, excising parathyroid tissue, protecting parathyroid tissue during thyroid surgery, and treating disorder(s) of a CSR-positive tissue in a subject.

### BACKGROUND OF THE INVENTION

Primary hyperparathyroidism (PHPT) is a common disorder that leads to significant morbidity and affects more than 100,000 persons each year in the United States. The parathyroid are small endocrine glands which control calcium level in the circulation in normal conditions. PHPT is caused by an intrinsic abnormality in the parathyroid glands, which secrete too much parathyroid hormone (PTH), causing an elevation of the serum calcium level. The excess hormone also drives calcium resorption from bone, which causes loss of bone mineral over time and can lead to osteoporosis. In addition, the elevated calcium is excreted by the kidneys, which can cause damage to the kidneys (nephrocalcinosis) and lead to the formation of kidney stones. The high circulating calcium is also damaging to blood vessels and predisposes a subject toward atherosclerotic diseases. There are also a number of neuropsychiatric symptoms caused by this disease including fatigue, depression, and a mental “fog”.

Currently, the only curative option for PHPT is surgically removing affected parathyroid gland(s). Most people have four parathyroid glands that are located adjacent to the much larger thyroid gland. Under normal conditions these glands are quite small (approximately the size of a lentil, i.e., 3 to 5 mm), which are not readily visible on current imaging tests. The most common cause of PHPT is development of a parathyroid adenoma (occurs in about 85% of cases) which causes enlargement of the affected one, two, or even three glands. In 5-10% of PHPT patients, all of the glands are affected due to hyperplasia. Collectively, multiple adenomas or parathyroid hyperplasia is termed “multi-gland disease,” because pathological analysis cannot reliably distinguish between multiple adenomas and hyperplasia. Accurate detection and identification of parathyroid glands are critical for the management of PHPT patients. Although parathyroid glands are normally found in the neck near the thyroid gland, in some cases, abnormal migration during embryogenesis causes them to be located anywhere from the base of the skull to the heart.

Conventionally, hyperparathyroidism treatment uses imaging to locate parathyroid glands preoperatively to direct the surgeon to the abnormal ones. The most commonly used modalities are computerized tomography (CT), ultrasound, and the nuclear medicine-sestamibi scan. Ultrasound has a sensitivity of approximately 75-80% and a positive predictive value (PPV) of 90-95% when performed by high volume, experienced centers. Ultrasound is limited by anatomic considerations, as it has poor ability to detect posteriorly located adenomas and does not detect ectopic glands. The 99mTc-sestamibi nuclear medicine study uses technetium labeled sestamibi as the tracer. 99mTc-sestamibi accumulates initially in both the thyroid and parathyroid but washes out of the thyroid more rapidly than the parathyroid. Delayed images are obtained after the tracer has washed out of the thyroid. Earlier techniques used simple planar scintigraphic imaging, while one current technique combines a single photon emission computed tomography scan with a computed tomography scan (SPECT-CT) for better anatomic resolution. Sestamibi/SPECT-CT has a sensitivity of 80-86% and a positive predictive value of 90-95%. The use of 4D CT, or dynamic CT has also become more popular in recent years. Pre, early phase, and late phase contrast-enhanced images are obtained in 4D CT. The region of the thyroid gland is then examined for small lesions that exhibit rapid washout to identify parathyroid tissues. The overall sensitivity of 4D CT ranges between 62% and 88%, with a PPV ranging between 84% and 90%. Despite the availability of multiple imaging modalities, in approximately 20% of cases, an abnormal gland cannot be located with current technology. In addition, current imaging studies are poor at detecting “multi-gland disease,” and small adenomas, usually <1 cm, are also often not visualized. These weaknesses are especially problematic in cases where the initial surgery was unsuccessful. Reoperation in search of a parathyroid gland in an unknown location is difficult and hazardous and often unsuccessful. Parathyroid glands can also be found in ectopic locations, often outside the neck. In these cases, imaging is vital to prevent fruitless neck exploration and to guide the resection of abnormally located glands.

Most surgeons continue to rely on visual identification which can take years of focused experience to develop adequately. Visual identification of parathyroid glands is also important in thyroidectomy to prevent inadvertent resection of normal parathyroid glands, which can result in hypoparathyroidism, an extremely morbid condition for affected patients.

Accurate detection and identification of parathyroid glands is critical for the management of PHPT patients. The present invention overcomes shortcomings in the art by providing ligands, fluorophores, probes, and compositions suitable for the detection of parathyroid tissue and/or calcium-sensing receptor (CSR) positive tissue, as well as methods of their use such as preoperative and/or intraoperative use to guide exploration or preserve normal glands during surgery.

### SUMMARY OF THE INVENTION

One aspect of the invention provides a radioisotope-labeled calcium sensing receptor (CSR) ligand comprising an aromatic ring, wherein the radioisotope is directly linked to the aromatic ring at one or more positions of the ring.

Another aspect of the invention provides a radioisotope-labeled CSR ligand comprising formula XX (etelcalcetide hydrochloride) directly linked to ¹⁸F.

Another aspect of the invention provides a labeled CSR ligand suitable for use as a positron emission tomography (PET) probe, fluorescence imaging (e.g., NIRF) probe and/or optical probe, comprising a CSR binding portion. In some embodiments, the ligand may be an antibody or antigen binding fragment thereof.

Another aspect of the invention provides a halogenated fluorophore comprising a radioisotope, capable of preferential uptake in thyroid and/or parathyroid tissue.

Also provided are PET probes, fluorescence imaging probes (e.g., NIRF probes), compositions, and pharmaceutical compositions comprising the ligands and/or fluorophores of the invention.

Additionally provided is a probe and/or composition of the invention, for use in the imaging, diagnosing, and/or guidance of treatment of a parathyroid disorder (e.g., primary hyperparathyroidism, secondary hyperparathyroidism, tertiary hyperparathyroidism), a thyroid disorder (e.g., thyroid cancer, goiter, thyroid nodules, Graves' disease), a cardiac disorder (e.g., hypertension), a kidney disorder (e.g., nephrocalcinosis, Rickets, proteinuria), a reproductive disorder (e.g., infertility, impaired embryonic or fetal growth), a lactation disorder (e.g., low milk production), a gastrointestinal disorder (e.g., pancreatitis, diabetes, diarrhea, impaired gut secretion), a bone disorder (e.g., osteoporosis), cancer (e.g., colon cancer), a neurological disorder (e.g., Alzheimer's, epilepsy), and/or a lung disorder (e.g., lung hypoplasia, lung hyperplasia).

A further aspect of the invention provides a method of carrying out a PET scan on a subject, comprising administering to the subject a ligand, fluorophore, probe, and/or composition of the invention.

Another aspect of the invention provides a method of imaging tissue comprising a CSR in a subject, comprising administering to the subject a ligand, fluorophore, probe, and/or composition of the invention.

Another aspect of the invention provides a method of imaging thyroid and/or parathyroid tissue in a subject, comprising administering to the subject a ligand, fluorophore, probe, and/or composition of the invention.

Another aspect of the invention provides a method of simultaneously carrying out a PET scan and fluorescence imaging (e.g., NIRF imaging) on a subject, comprising administering to the subject a ligand, fluorophore, probe, and/or composition of the invention.

Another aspect of the invention provides a method of identifying parathyroid tissue in a subject, comprising carrying out a PET scan and/or fluorescence imaging (e.g., NIRF imaging) on the subject using a ligand, fluorophore, probe, or composition of the invention, wherein the PET scan and/or fluorescence imaging identifies presence of parathyroid tissue.

Another aspect of the invention provides a method of removing hyperplastic and/or ectopic parathyroid tissue in a subject, comprising: (a) carrying out a PET scan and/or fluorescence imaging (e.g., NIRF imaging) on the subject using a ligand, fluorophore, probe, and/or composition of the invention, wherein the PET scan and/or fluorescence imaging identifies presence of parathyroid tissue; (b) identifying present parathyroid tissue that is hyperplastic and/or ectopic; and (C) surgically excising the identified hyperplastic and/or ectopic parathyroid tissue, thereby removing the hyperplastic and/or ectopic parathyroid tissue.

Another aspect of the invention provides a method of guiding surgery for the removal of parathyroid tissue in a subject, comprising carrying out a PET scan and/or fluorescence imaging (e.g., NIRF imaging) on the subject using a ligand, fluorophore, probe, and/or composition of the invention, wherein the PET scan and/or fluorescence imaging identifies presence of parathyroid tissue.

Another aspect of the invention provides a method of guiding surgery for the protection of parathyroid tissue during thyroid and/or other neck surgery in a subject, comprising carrying out a PET scan and/or fluorescence imaging (e.g., NIRF imaging) on the subject using a ligand, fluorophore, probe, and/or composition of the invention, wherein the PET scan and/or fluorescence imaging identifies presence of parathyroid tissue.

Another aspect of the invention provides a method of determining target regions for surgical removal of parathyroid tissue of a subject (e.g., a subject with hyperparathyroidism or a subject at risk for or suspected to have or develop hyperparathyroidism), comprising (a) carrying out a PET scan and/or fluorescence imaging (e.g., NIRF imaging) on the subject 15 using a ligand, fluorophore, probe, and/or composition of the invention, wherein the PET scan and/or fluorescence imaging identifies presence of parathyroid tissue; and (b) identifying one or more regions(s) of the subject comprising the presence of ectopic and/or hyperplastic parathyroid tissue, wherein the presence of ectopic and/or hyperplastic parathyroid tissue in the one or more region(s) indicates the region(s) as a target region of the subject for surgical removal of parathyroid tissue.

Another aspect of the invention provides a method of treating hyperparathyroidism in a subject (e.g., primary, secondary, and/or tertiary hyperthyroidism), comprising determining the suitability of a subject with hyperparathyroidism or a subject at risk for or suspected to have or develop hyperparathyroidism to surgical removal of parathyroid tissue by carrying out a PET scan and/or fluorescence imaging (e.g., NIRF imaging) on the subject using a ligand, fluorophore, probe, or composition of the invention, wherein the PET scan and/or fluorescence imaging identifies presence of parathyroid tissue, and treating the hyperparathyroidism based on the results of the PET scan and/or fluorescence imaging.

Another aspect of the invention provides a method of treating a disorder of a CSR-positive tissue in a subject, comprising determining suitability of a subject with the disorder or a subject at risk for or suspected to have or develop the disorder to treatment thereof by carrying out a PET scan and/or fluorescence imaging (e.g., NIRF imaging) on the subject using a ligand, fluorophore, probe, or composition of the invention, wherein the PET scan and/or fluorescence imaging identifies presence of CSR-positive tissue, and treating the disorder based on the results of the PET scan and/or fluorescence imaging.

In some embodiments, the subject may be a pre-operative subject. In some embodiments, the subject may be an intra-operative subject (e.g., wherein the subject is undergoing surgery (e.g., explorative surgery).

These and other aspects of the invention are set forth in more detail in the description of the invention below.

### BRIEF DESCRIPTION OF THE DRAWINGS

FIG. 1 shows a table and histology images of patient tissue staining of CSR expression in hyperparathyroidism and parathyroid glands (n=10 for each type of patient specimen).

FIG. 2 shows two schematics of representative paths for generating a synthetic ¹⁸F-cinacalcet. Route A (top), shows a representative route of synthesizing ¹⁸F-cinacalcet through direct C—H radiofluorination. Route B (bottom) shows two synthetic routes (Path A and Path B) to label [¹⁸F]-CF₃-cinacalcet at the CF₃ group.

FIG. 3 shows a schematic of photoredox radiofluorination that could efficiently introduce ¹⁸F into boc protected cinacalcet (FIG. 3 panel A), which correlates well with the standard prepared through route (FIG. 3 panel B). FIG. 3 panel C shows graphs of quality control of ¹⁸F labeled boc-cinacalcet. The unreacted boc-cinacalcet could be well separated from the ¹⁸F labeled product.

FIG. 4A shows a graph of ¹⁸F-cinacalcet demonstrated stability in vitro.

FIG. 4B shows a graph of ¹⁸F-cinacalcet stability in vivo non-human primates.

FIG. 5 shows images of (FIG. 5 panel A) small animal PET/CT scan demonstrating the accumulation of ¹⁸F-cinacalcet in parathyroid region; and (FIG. 5 panel B) autoradiography and pathology staining of neighboring slides demonstrating that ¹⁸F-cinacalcet is localized at the CSR positive parathyroid gland instead of thyroid.

FIG. 6 shows two schematics, where top box “A” shows a representative synthetic route for precursor and standard preparation for T700, and bottom box “B” shows the structure of T800 dye and related precursors.

FIG. 7 shows a representative route of synthesizing [¹⁸F]T700 dye through C—H radiofluorination (route “A”), and a representative route of synthesizing [¹⁸F]T800 dye through deoxy-radiofluorination (route “B”). T700 could also be synthesized through deoxyradiofluorination; T800 could also be made through CH fluorination.

FIG. 8 shows images of (FIG. 8 panel A) fluorescence imaging parameters for T700 and T800 dual channel imaging; (FIG. 8 panel B) dual channel imaging indicating T700 was localized in thyroid gland and T800 was localized in parathyroid gland; and (FIG. 8 panel C) fluorescence images indicating autofluorescence from mice 1-3 was negligible compared with mouse 3 (injected with T800) imaged at T800 channel. Mouse 2 was injected with T700 and mouse 1 was injected with saline.

FIG. 9 shows the synthesized ¹⁸F-T800 (FIG. 9 panel A) and ¹⁸F-T700 (FIG. 9 panel B) synthesized using photoredox labeling method. The products correlate well with cold standards.

FIG. 10 shows images of a transplanted PHPT model, which could be visualized by T800 (left panel). Image guided surgery showed high contrast between PHPT and nearby tissue (right panel).

FIG. 11 shows PET images of ¹⁸F-cinacalcet PET of native parathyroid of a Rhesus Macaque.

FIG. 12 shows a schematic of (FIG. 12 panel A) traditional PET/NIRF probe construction and (FIG. 12 panel B) 2-in-1 radioactive fluorescent dye PET/NIRF probe construction.

FIG. 13 shows a schematic representative path for the chemical synthesis of [¹⁹F]F-ZW-cinacalcet and the precursor for photoredox reaction (FIG. 13 panel A) and the photoredox radiolabeling of Boc-cinacalcet through direct C—H fluorination (FIG. 13 panel B).

FIG. 14 shows the results of CSR expression by western blot in various cell lines (FIG. 14 panel A). Cell uptake and specific blocking assays of [¹⁸F]F-ZW-cinacalcet are shown in FIG. 14 panel B.

FIG. 15 shows representative coronal PET images (FIG. 15 panel A) and quantitative analysis (FIG. 15 panel B) in rats injected with [¹⁸F]F-cinacalcet at 0.5, 1 and 2 h p.i. Arrows indicate parathyroid.

FIG. 16 shows representative coronal, sagittal, transverse PET/CT images (FIG. 16 panel A) and 3D volume-rendered PET/CT images (FIG. 16 panel B) in rats injected with [¹⁸F]F-cinacalcet at 0.5 h. Position line and filled arrows indicate parathyroid, open arrows indicate trachea.

FIG. 17 shows representative coronal, dynamic PET images and quantitative analysis in rats injected with [¹⁸F]F-cinacalcet at 0-60 min. Arrows indicate parathyroid.

FIG. 18 shows representative transverse, dynamic PET images (FIG. 18 panel A), merged PET/CT images (FIG. 18 panel B) and quantitative analysis (FIG. 18 panel C) in rats injected with [¹⁸F]F-ZW-cinacalcet at 0-60 min. Filled arrows indicate heart and open arrows indicate lung.

FIG. 19 shows an anatomical diagram and excised tissues of regional larynx and tracheal tissues containing thyroid and parathyroid (FIG. 19 panel A), with matched autoradiography (FIG. 19 panel B) and IHC staining (FIG. 19 panel C) of CSR in rats injected with [¹⁸F]F-cinacalcet, and quantitative analysis (FIG. 19 panel D). Circles indicate thyroid containing parathyroid, arrows indicate parathyroid, **P<0.01.

FIG. 20 shows IHC staining of CSR in paraffin embedded tissue sections. Circles indicate thyroid containing parathyroid, arrows indicate parathyroid.

FIG. 21 shows autoradiography, with matched IHC staining and HE staining of CSR in paraffin embedded tissue sections.

FIG. 22 shows clinical PET/MRI imaging of parathyroid in nonhuman primates, arrows indicate parathyroid.

FIG. 23 shows HE staining of kidney, liver, and heart in mice upon administration of an overdose of [¹⁹F]F-cinacalcet at different time points. Basic structures of the glomerulus, renal tubulus, liver lobule central vein, and cardiac muscle fibers are intact. Scale bars=200 μm.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” (and grammatical variants), as applied to the compositions of this invention, means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “materially altered,” as applied to a composition, refers to an increase or decrease in the therapeutic effectiveness of the composition of at least about 20% or more as compared to the effectiveness of a composition consisting of the recited components.

The terms “substantially retain” and/or “not substantially alter” as used herein in reference to a property (e.g., structure, function, or other measurable characteristic) of a compound, refer to maintaining said property “substantially the same” as a comparison (e.g., a control), wherein at least about 75%, 85%, 90%, 95%, 96%, 97%, 98%, 99% or 100% of the property (e.g., structure, functionality and/or other measurable characteristic) is retained.

The terms “treat” or “treating” or “treatment” refer to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, etc., as would be well known in the art.

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

A “treatment effective” amount, “effective amount,” or “therapeutic amount” as used herein is an amount that is sufficient to provide some improvement or benefit to the subject. Alternatively stated, a “treatment effective amount,” “effective amount,” or “therapeutic amount” is an amount that will provide some alleviation, mitigation, decrease or stabilization in at least one clinical symptom in the subject. Those skilled in the art will appreciate that the therapeutic effects need not be complete or curative, as long as some benefit is provided to the subject. The effective amount may vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutic amount in any individual case can be determined by one of ordinary skill in the art by reference to the pertinent texts and literature and/or by using routine experimentation. (See, for example, Remington, The Science and Practice of Pharmacy (20th ed. 2000)).

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, sterile pyrogen-free water and sterile pyrogen-free physiological saline solution.

The term “administering” or “administration” of a composition of the present invention to a subject includes any route of introducing or delivering to a subject a compound to perform its intended function (e.g., for use in PET and/or fluorescence imaging, e.g., for the guidance of surgery).

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human.

A “subject in need” of the methods of the invention can be any subject known or suspected to have a thyroid and/or parathyroid disorder and/or any CSR-expressing tissue disorder and/or an illness to which imaging and/or surgery may provide beneficial health effects, or a subject having an increased risk of developing the same).

A “sample”, “biological sample”, and/or “ex vivo sample” of this invention can be any biological material, such as a biological fluid, an extract from a cell, an extracellular matrix isolated from a cell, a cell (in solution or bound to a solid support), a tissue, a tissue homogenate, and the like as are well known in the art. The terms “amino acid sequence.” “polypeptide,” “peptide” and “protein” may be used interchangeably to refer to polymers of amino acids of any length. The terms “nucleic acid,” “nucleic acid sequence,” and “polynucleotide” may be used interchangeably to refer to polymers of nucleotides of any length. As used herein, the terms “nucleotide sequence,” “polynucleotide,” “nucleic acid sequence,” “nucleic acid molecule” and “nucleic acid fragment” may refer to a polymer of RNA, DNA, or RNA and DNA that is single- or double-stranded, optionally containing synthetic, non-natural and/or altered nucleotide bases. As used herein, the term “binding portion” refers to the portion (e.g., fragment) of a molecule which binds to another molecule (e.g., a target). For example, a “CSR binding portion” as used herein refers to a portion of a molecule (e.g., a ligand, e.g. a fluorophore) which is capable of binding to CSR. The binding portion may be, e.g., isolated from a molecule or compound, synthetically generated de novo, and/or comprised within a larger molecule (e.g., a ligand, a fluorophore, an antibody, etc.).

As used herein, the term “antigen” refers to a molecule capable of inducing the production of immunoglobulins (e.g., antibodies). A molecule capable of antibody and/or immune response stimulation may be referred to as antigenic and/or immunogenic, and can be said to have the ability of antigenicity/immunogenicity. The binding site for an antigen comprised in an antibody may be referred to as an antigen binding portion. An antigen binding portion may be, e.g., isolated from an antibody, synthetically generated de novo, and/or comprised within a larger molecule (e.g., an antibody or fragment thereof).

As used herein, the term “antibody” includes intact immunoglobulin molecules as well as active fragments thereof, such as Fab, F(ab′)2, and Fc, which are capable of binding the epitopic determinant of an antigen (i.e., antigenic determinant). Antibodies that bind the polypeptides of this invention are prepared using intact polypeptides and/or fragments containing small peptides of interest as the immunizing antigen. The polypeptide or fragment used to immunize an animal can be derived from enzymatic cleavage, recombinant expression, isolation from biological materials, synthesis, etc., and can be conjugated to a carrier protein, if desired. Commonly used carriers that are chemically coupled to peptides and proteins for the production of antibody include, but are not limited to, bovine serum albumin, thyroglobulin and keyhole limpet hemocyanin. The coupled peptide or protein is then used to immunize a host animal (e.g., a mouse, rat, goat, sheep, human or rabbit). The polypeptide or peptide antigens can also be administered with an immunostimulatory agent, as described herein and as otherwise known in the art.

The terms “antibody” and “antibodies” as used herein refer to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, and/or can be a chimeric or humanized antibody. See. e.g., Walker et al., Molec. Immunol. 26:403-11 (1989). The antibodies can be recombinant monoclonal antibodies produced according to the methods disclosed in U.S. Pat. Nos. 4,474,893 or 4,816,567. The antibodies can also be chemically constructed according to the method disclosed in U.S. Pat. No. 4,676,980. The antibody can further be a single chain antibody (scFv) or bispecific antibody.

Techniques for the production of chimeric antibodies or humanized antibodies by splicing mouse antibody genes to human antibody genes to obtain a molecule with appropriate antigen specificity and biological activity can be used (Morrison et al. 1984. Proc. Natl. Acad. Sci. 81:6851-6855; Neuberger et al. 1984. Nature 312:604-608; Takeda et al. 1985. Nature 314:452-454). Alternatively, techniques described for the production of single chain antibodies can be adapted, using methods known in the art, to produce single chain antibodies specific for the polypeptides and/or fragments and/or epitopes of this invention. Antibodies with related specificity, but of distinct idiotypic composition, can be generated by chain shuffling from random combinatorial immunoglobulin libraries (Burton 1991. Proc. Natl. Acad. Sci. 88:11120-3).

As used herein with respect to proteins, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to proteins, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the antigen-binding antibody).

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

As used herein, by “isolate” or “purify” (or grammatical equivalents) a fragment, it is meant that the fragment is at least partially separated from at least some of the other components in the starting material.

Non-limiting examples of an antibody or active antibody fragment include a monoclonal antibody or fragment thereof, a chimeric antibody or fragment thereof, a CDR-grafted antibody or fragment thereof, a humanized antibody or fragment thereof, an Fc, a Fab, a Fab′, a F(ab′)2, a Fv, a disulfide linked Fv, a single chain antibody (scFv), a single domain antibody (dAb), a diabody, a multispecific antibody (e.g., a bispecific antibody) or fragment thereof, an anti-idiotypic antibody or fragment thereof, a bifunctional hybrid antibody or fragment thereof, a functionally active epitope-binding antibody fragment, an affibody, a nanobody, and any combination thereof.

Active antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281).

Monoclonal antibodies can be produced in a hybridoma cell line according to the technique of Kohler and Milstein (Nature 265:495-97 (1975)). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in bacterial cell such as E. coli by recombinant techniques known to those skilled in the art. See. e.g., W. Huse, (1989) Science 246:1275-81.

### Compounds and Compositions

Positron emission tomography (PET) is a powerful and rapidly developing technology that is used for quantitative in vivo measurement of site-specific chemical reactions, their spatial distributions and metabolic perturbations, and ensuing biological processes with a high degree of accuracy and sensitivity.

Fluorescence imaging has limited tissue penetration but, has been demonstrated to be a valuable tool to increase sensitivity for intraoperative lesion detection (invasive surgery). The present invention provides novel PET probes, as well as highly sensitive and specific PET/NIRF imaging techniques that may efficiently detect the parathyroid gland preoperatively, and also guide subsequent localization during surgery. While not wishing to be bound to theory, this combined technique may significantly reduce surgery times and improve the surgical yield.

One problem the invention addresses is determining the identity of suspicious tissues and the location of parathyroid glands (rather than distinguishing PHPT from normal parathyroid). In fact, normal parathyroid glands are quite small (3 to 5 mm), which are not readily visible on current imaging tests. In PHPT, one or more of the glands enlarge as they become autonomous in parathyroid hormone secretion. Normal parathyroid and hyperparathyroidism could be easily distinguished by their size difference; the challenging question is to identify their locations. Despite the availability of multiple imaging modalities, in approximately 20% of cases, an abnormal gland cannot be located with current technology including ultrasound, ^{99m}Tc-sestamibi, or 4D CT. This becomes a real problem especially for multi-gland disease and small adenomas, which could lead to re-operation and fruitless neck exploration. Here, a CSR-specific radio-pharmaceutical [¹⁸F]F-ZW-cinacalcet as a potential novel PET agent for parathyroid imaging was developed. This imaging agent is highly sensitive for parathyroid, as demonstrated in both rodents and non-human primates. This imaging approach can guide a surgeon performing surgery especially when the glands are not in normal locations. In another situation, a small nodule (or nodules) may be seen near the thyroid on ultrasound, but its identity as a parathyroid adenoma as opposed to a lymph node or focus of ectopic thyroid tissue may not be readily apparent. [¹⁸F]F-ZW-cinacalcet can help identify the suspicious tissue origin, which would avoid unnecessary surgery. The development of [¹⁸F]F-ZW-cinacalcet also represents a successful application of photoredox labeling in PET probe development.

CSR is a transmembrane G-protein coupled receptor which responds to the calcium concentration in the circulation. CSR is expressed primarily in the parathyroid and kidney, although a wide variety of tissues express this receptor (Brown, E. M. and Macleod, R. J., 2001 Physiol. Rev. 81:239-297). Two calcimimetic drugs are clinically available that bind CSR: cinacalcet, an orally available small molecule; and etelcalcetide, a synthetic peptide given intravenously. In addition, antibodies to CSR are also available.

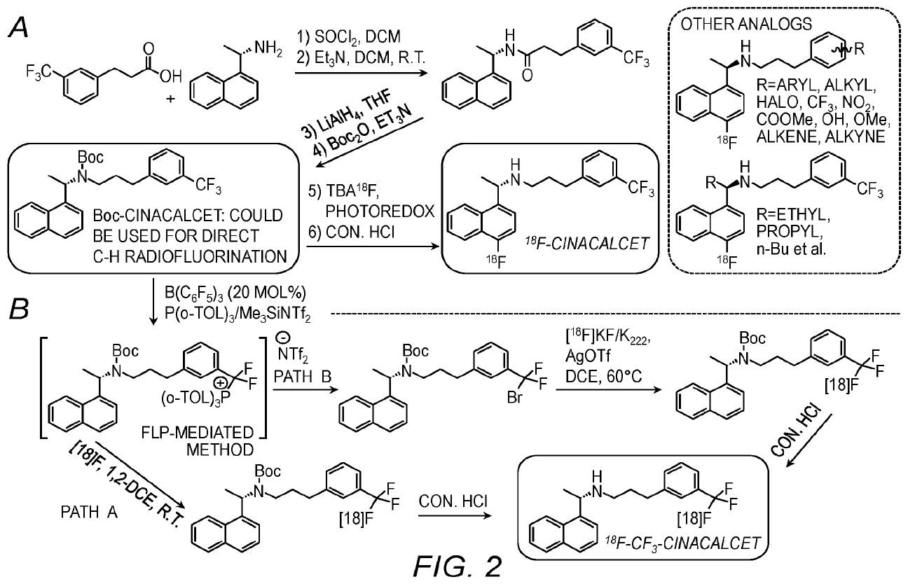

Synthesis of the compounds of the invention may be carried out by methods known in the art and as described herein. In some embodiments, the synthetic route comprises radio-fluorination (e.g., S_{N}Ar radiofluorination, metal catalyzed radiofluorination, iodonium radiofluorination, laser induced radiofluorination, arene C—H radiolabeling) such as described in Chen et al. 2019 Science 364(6446):1170-1174, incorporated herein by reference. In some embodiments, the synthetic route may comprise any route as described herein and as shown, for example, in FIGS. 2, 3, 6, and 7.

Accordingly, one aspect of the invention relates to a radioisotope-labeled calcium sensing receptor (CSR) ligand comprising an aromatic ring, wherein the radioisotope is directly linked to the aromatic ring at one or more positions of the ring.

The radioisotope may be any radioisotope label which would not substantially alter the biological activity (e.g., CSR-binding) of the ligand. In some embodiments, the radioisotope may be ¹⁸F and/or ¹¹C. In some embodiments, the ¹¹C may be ¹¹CN, ¹¹COOH, and/or ¹¹CH₃.

A radioisotope-labeled CSR ligand of the present invention may comprise any type of aromatic ring, including but not limited to, heteroaromatic rings, arene rings, phenyl rings, and the like. In some embodiments, the aromatic ring may be an arene ring. In some embodiments, the arene ring may be a naphthalene ring and/or phenyl ring.

The radioisotope of a radioisotope-labeled CSR ligand of the present invention may be directly linked to the aromatic ring of the ligand in any position of the ring, through any process, including but not limited to, via CH fluorination and/or through nucleophilic aromatic S_{N}Ar (addition-elimination) mechanism. In some embodiments, the ligand may comprise ¹⁸F and/or ¹¹C directly linked to an arene ring at position 1, 2, 3, 4, 5 or others. In some embodiments, the ligand may comprise ¹⁸F and/or ¹¹C directly linked to a naphthalene ring at position 1, 2, 3, 4, 5 or others. In some embodiments, the ligand may comprise ¹⁸F and/or ¹¹C directly linked to a naphthalene ring at position 2 and/or 4. In some embodiments, the ligand may comprise ¹⁸F directly linked to a naphthalene ring at position 2 and/or 4. In some embodiments, the ligand may comprise ¹¹C directly linked to a naphthalene ring at position 2 and/or 4.

In some embodiments, the radioisotope-labeled CSR ligand of the present invention may comprise formula II, wherein Ar is substituted aryl and Y is alkyl.

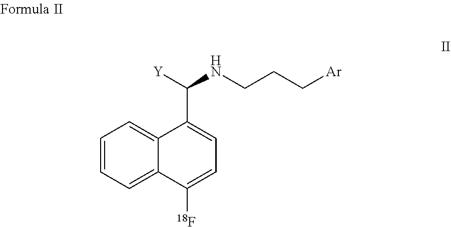

The Ar may be any aryl, including but not limited to the groups shown below, wherein X is O, S, NH, or CH₂, and R is aryl, alkyl, halo, CF₃, NO₂, COOMe, OH, OMe, alkene, or alkyne.

### Non-Limiting Examples of Ar Groups

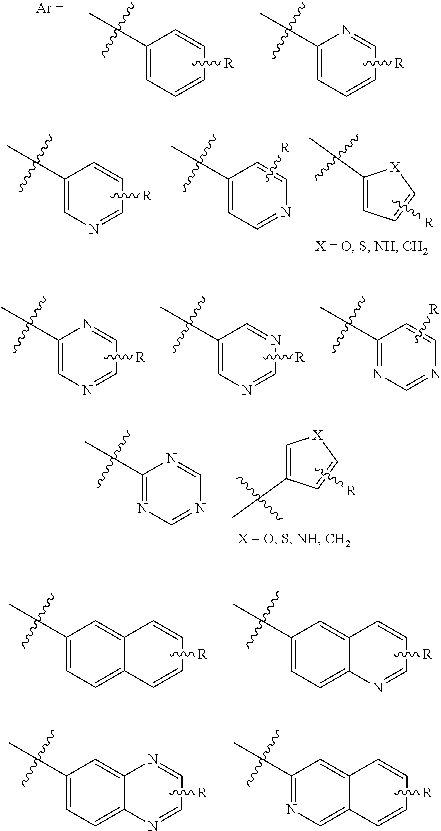

In some embodiments. the radioisotope-labeled CSR ligand of the present invention may comprise Formula III.

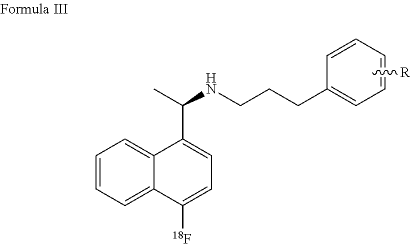

In some embodiments. the radioisotope-labeled CSR ligand of the present invention may comprise any one of Formulas Ia-Ih.

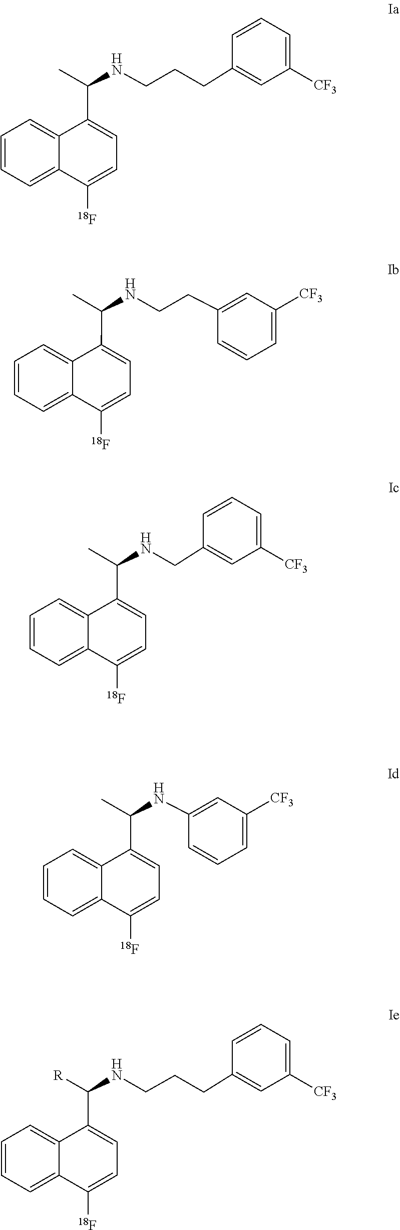

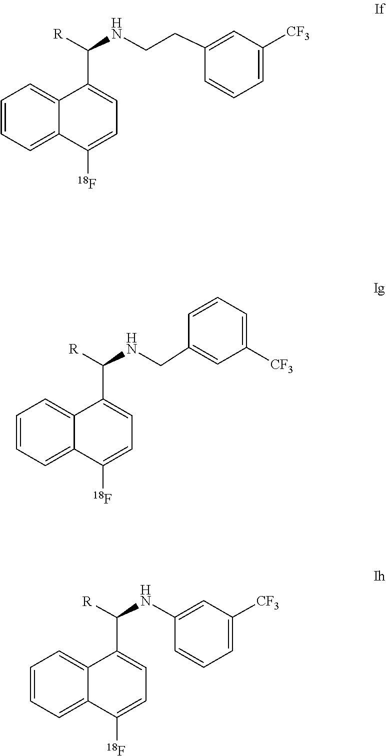

In some embodiments. the radioisotope-labeled CSR ligand of the present invention may comprise Formula I.

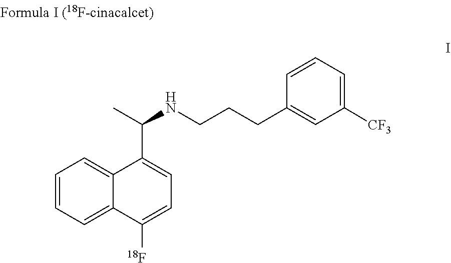 [SMILES: C[C@@H](NCCCc1cccc(C(F)(F)F)c1)c1ccc([18F])c2ccccc12]

In some embodiments. the Ar group may be linked to the compound by an aliphatic chain linker or a chain comprising heteroatoms. In some embodiments. the linker may be an aliphatic chain. In some embodiments, the linker may further comprise additional atoms such as but not limited to oxygen, sulfur, nitrogen, or the like.

In some embodiments, the radioisotope-labeled CSR ligand of the present invention may comprise any one of Formulas VIII, IX, and/or X.

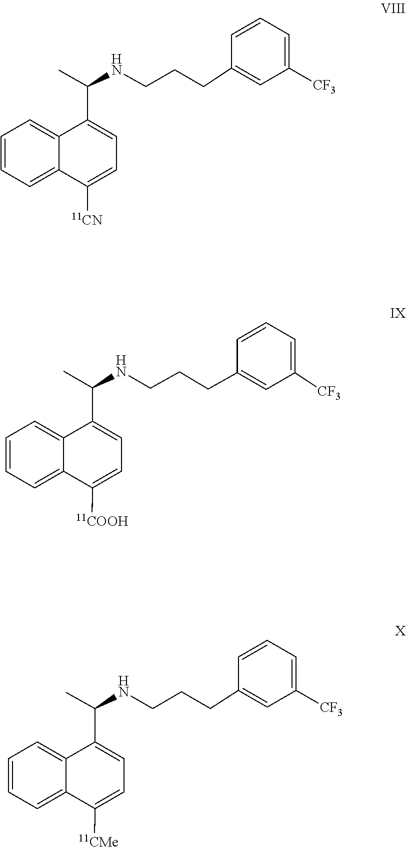

In some embodiments, a ligand of the present invention may comprise a radioisotope-labeled calcium sensing receptor (CSR) ligand comprising an aromatic ring, wherein the ligand does not comprise formula XXI.

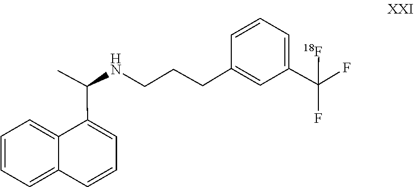 [SMILES: C[C@@H](NCCCc1cccc(C(F)(F)[18F])c1)c1cccc2ccccc12]

In some embodiments, the radioisotope-labeled CSR ligand of the present invention may comprise any radioisotope-labeled derivative of the drug NPS-2143 (SB-262470A). Non-limiting examples of radioisotope-labeled CSR ligands of the present invention derived from the drug NPS-2143 include any one of Formulas IV, V, VI, and/or VII (NPS-2143 derivatives).

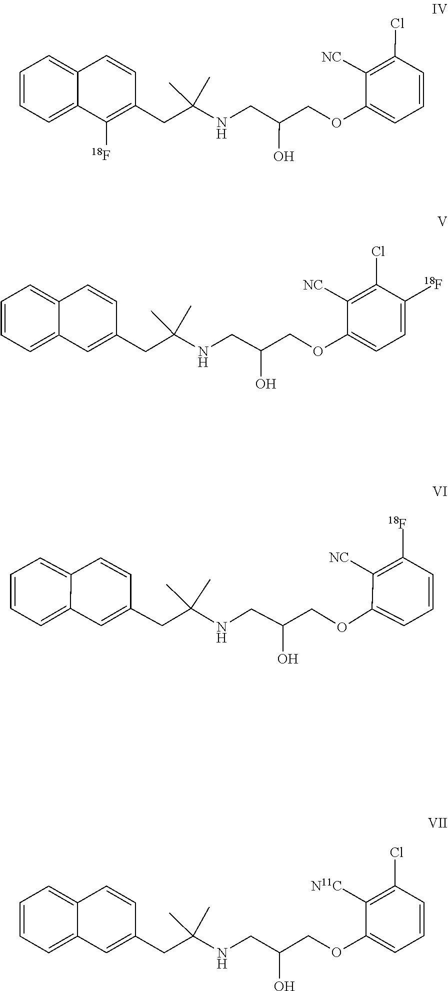

In some embodiments, the radioisotope-labeled CSR ligand of the present invention may comprise any radioisotope-labeled derivative of the drug evocalcet (CAS No. 870964-67-3. In some embodiments, the radioisotope-labeled CSR ligand of the present invention may comprise Formula XI, wherein the radioisotope may be directly linked to the naphthalene at position 4.

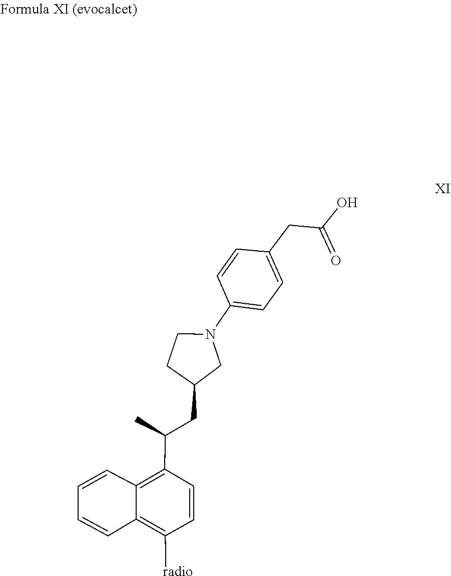

In some embodiments, the radioisotope-labeled CSR ligand of the present invention may comprise any one of Formulas XII, XIII, and/or XIV.

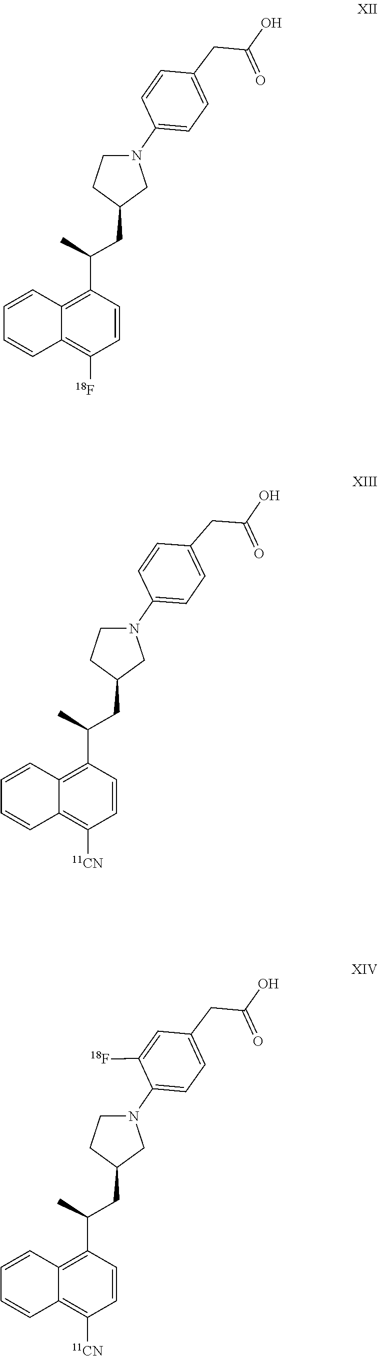

In some embodiments, the radioisotope-labeled CSR ligand of the present invention may comprise any radioisotope-labeled derivative of the drug SB-423562 (CAS No. 351490-27-2; formula XV).

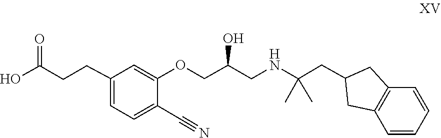 [SMILES: CC(C)(CC1Cc2ccccc2C1)NC[C@H](O)COc1cc(CCC(=O)O)ccc1C#N]

Non-limiting examples of radioisotope-labeled CSR ligands of the present invention derived from the drug SB-423562 include any one of formulas XVI, XVII, XVIII, and XIX.

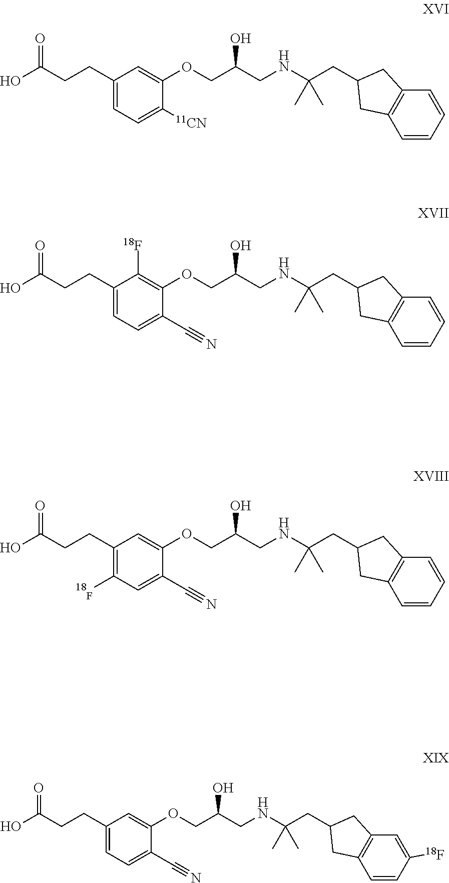

In some embodiments, the radioisotope-labeled CSR ligand of the present invention may comprise any radioisotope-labeled derivative of the drug etelcalcetide hydrochloride (CAS No. 1334237-71-6; formula XX). In some embodiments, the radioisotope-labeled CSR ligand of the present invention may comprise formula XX (etelcalcetide hydrochloride) directly linked to ¹⁸F.

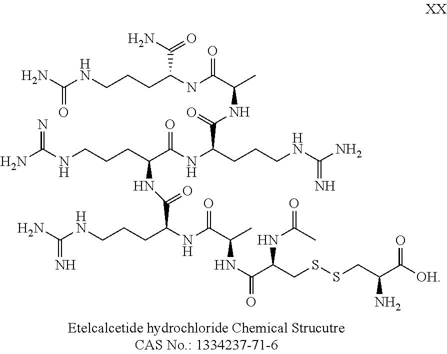

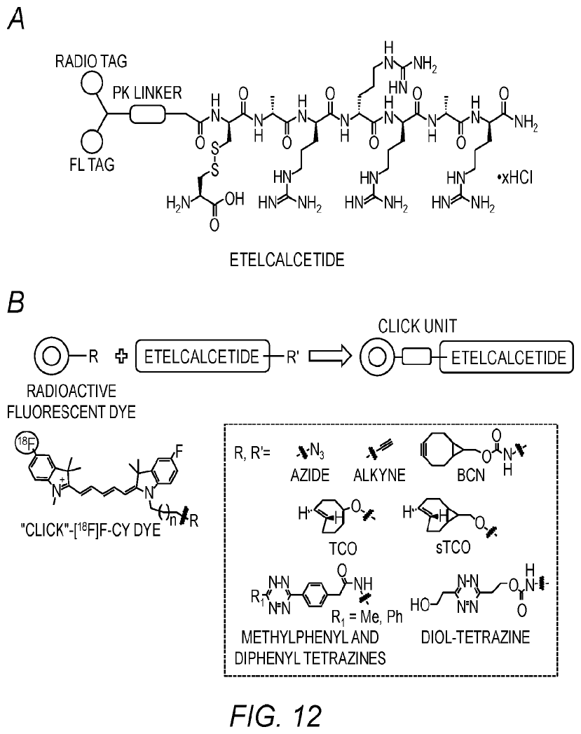

The structures provided herein of labeled cinacalcet, NPS-2143, SB-423562, evocalcet, etelcalcetide hydrochloride, and derivatives thereof are examples of the ligands of the present invention and are not intended to be limiting. Other radioisotope labels and other positions of direct linkage of the label are also contemplated. For example, in some embodiments, etelcalcetide hydrochloride may also comprise ⁶⁴Cu, ⁶⁸Ga, and/or ⁸⁹Zr labeling, for example, through chelation, and as diagramed in FIG. 12.

Another aspect of the present invention provides a labeled calcium sensing receptor (CSR) ligand suitable for use as a positron emission tomography (PET) probe, fluorescence imaging (e.g., NIRF) probe and/or optical probe, comprising a CSR binding portion.

The CSR ligand suitable for use as a PET probe, fluorescence probe, and/or optical probe may be any type of ligand which selectively binds to CSR (e.g., comprises a CSR binding portion.

The label of the labeled CSR ligand of the present invention may be any label which does not substantially alter the biological activity (e.g., CSR-binding) of the ligand. In some embodiments, the label may be a fluorescent dye (e.g., a near-infrared (NIR) dye or NIR-II dye). In some embodiments, the label may be a radioisotope. For example, in some embodiments the label may be, but is not limited to ¹⁸F, ¹¹C, ⁶⁸Ga, ⁸⁹Zr, ⁶⁴Cu, ⁸⁷Y, ¹²⁴I, ⁴⁴Sc, and the like, or any combination thereof. In some embodiments, the label may be ¹⁸F and/or ¹¹C. In some embodiments, the ¹¹C may be ¹¹CN, ¹¹COOH, and/or ¹¹CH₃.

In some embodiments, the ligand may be an antibody or antigen binding fragment thereof. For example, in some embodiments, the antibody or antibody fragment may be, but is not limited to, a monoclonal antibody or fragment thereof, a chimeric antibody or fragment thereof, a CDR-grafted antibody or fragment thereof, a humanized antibody or fragment thereof, an Fc, a Fab, a Fab′, a F(ab′)2, a Fv, a disulfide linked Fv, a single chain antibody (scFv), a single domain antibody (dAb), a diabody, a multispecific antibody (e.g., a bispecific antibody) or fragment thereof, an anti-idiotypic antibody or fragment thereof, a bifunctional hybrid antibody or fragment thereof, a functionally active epitope-binding antibody fragment. an affibody, a nanobody, and any combination thereof.

In some embodiments, the antibody may be a known antibody with antigenic specificity for CSR (e.g., an anti-CSR antibody, also referred to as anti-CaSR antibody). In some embodiments, the antibody may be, but is not limited to, monoclonal anti-CSR antibody clone 5C10, ADD, 3F12, 611825, EPR24050-59, 6D4, and/or HL1499. In some embodiments, the antibody may be de novo generated.

Another aspect of the present invention provides a halogenated fluorophore comprising a radioisotope, capable of preferential uptake in thyroid and/or parathyroid tissue.

The radioisotope may be any radioisotope label which would not substantially alter the biological activity (e.g., thyroid and/or parathyroid uptake) of the ligand. In some embodiments, the radioisotope may be ¹⁸F and/or ¹¹C. In some embodiments, the radioisotope is ¹⁸F.

In some embodiments, the halogenated fluorophore may comprise formula XXII (18F-T700).

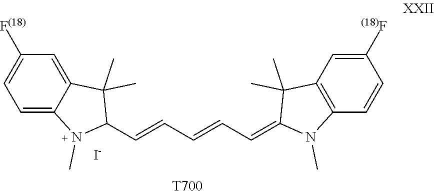

In some embodiments, the halogenated fluorophore may comprise formula XXIII (¹⁸F-T800).

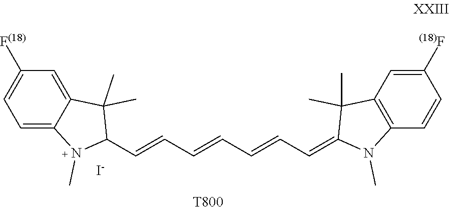

In some embodiments, the labeled CSR ligands and/or fluorophores of the present invention may have a serum stability of at least 70% or higher (e.g., at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or higher, or any value or range therein). For example, in some embodiments, a labeled CSR ligand (e.g., a radioisotope-labeled CSR ligand), and/or a halogenated fluorophore of the present invention may have serum stability of at least 70%, at least 85%, or at least 90%. Serum stability of a ligand and/or fluorophore of the present invention may be measured by any standard method known in the art, including but not limited to, by co-incubation with solution containing serum proteins, such as described in Qu et al. 2019 Anim. Cells Syst. (Seoul) 23:155-163, incorporated herein by reference. In some embodiments, the labeled CSR ligands and/or fluorophores of the present invention may a metabolic stability of at least 50% or higher (e.g., at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or higher, or any value or range therein). For example, in some embodiments, a labeled CSR ligand (e.g., a radioisotope-labeled CSR ligand), and/or a halogenated fluorophore of the present invention may have a metabolic stability of at least 50%, at least 65%, at least 70%, at least 80%, at least 85%, or at least 90%. Metabolic stability of a ligand and/or fluorophore of the present invention may be measured by any standard method known in the art, including but not limited to, by HPLC analysis such as described in Qu et al. 2019 Anim. Cells Syst. (Seoul) 23:155-163, incorporated herein by reference.

Another aspect of the invention relates to a PET probe comprising a ligand or fluorophore of the invention.

Another aspect of the invention relates to an optical probe comprising a ligand or fluorophore of the invention. In some embodiments, the optical probe may be a fluorescence probe. In some embodiments, the probe may be a dual tracer (e.g., an optical probe and a PET probe, e.g., a fluorescence probe and a PET probe).

Another aspect of the invention relates to a fluorescence imaging probe (e.g., NIRF probe) comprising a ligand or fluorophore of the invention. In some embodiments, a fluorescence imaging probe of the present invention may be a near-infrared fluorescence (NIRF) probe. In some embodiments, a fluorescence imaging probe of the present invention may be a conventional (visible light) fluorescence probe (e.g., excitation at about 300 nanometers (nm) to about 850 nm wavelength). In some embodiments, a fluorescence imaging probe of the present invention may be a shortwave infrared (SWIR) fluorescence probe. In some embodiments, the probe may be a dual tracer (e.g., an optical probe and a PET probe, a fluorescence probe and a PET probe, a NIRF probe and a PET probe).

Another aspect of the invention relates to a composition comprising a ligand, fluorophore, and/or probe of the present invention and a pharmaceutically acceptable carrier.

In some embodiments, the present invention provides a pharmaceutical composition comprising a ligand, fluorophore, and/or probe of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and will preferably be in solid or liquid particulate form.

By “pharmaceutically acceptable” it is meant a material that is not toxic or otherwise undesirable, i.e., the material may be administered to a subject without causing any undesirable biological effects.

In some embodiments, a composition of the present invention comprising a radioisotope-labeled ligand and/or fluorophore of the present invention may have a radio purity of at least 70% or higher (e.g., at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or higher, or any value or range therein). For example, in some embodiments, a composition comprising a radioisotope-labeled CSR ligand and/or a halogenated fluorophore of the present invention may have a radio purity of at least 70%, at least 75%, at least 80%, at least 85%, at least 90%, at least 95%, or at least 98% or higher.

In some embodiments, a ligand, fluorophore, probe and/or composition of the present invention may be for use in imaging, diagnosing, and/or guidance of treatment of a disorder. Non-limiting examples of disorders of use in the present invention include a parathyroid disorder (e.g., primary hyperparathyroidism, secondary hyperparathyroidism, tertiary hyperthyroidism), a thyroid disorder (e.g., thyroid cancer, goiter, thyroid nodules, Graves' disease), a cardiac disorder (e.g., hypertension), a kidney disorder (e.g., nephrocalcinosis, Rickets, proteinuria), a reproductive disorder (e.g., infertility, impaired embryonic or fetal growth), a lactation disorder (e.g., low milk production), a gastrointestinal disorder (e.g., pancreatitis, diabetes, diarrhea, impaired gut secretion), a bone disorder (e.g., osteoporosis), cancer (e.g., colon cancer), a neurological disorder (e.g., Alzheimer's, epilepsy), and/or a lung disorder (e.g., lung hypoplasia, lung hyperplasia). In some embodiments, a ligand, fluorophore, probe, and/or composition of the present invention may be for use in imaging, diagnosing, and/or guidance of treatment of a parathyroid disorder. In some embodiments, a ligand, fluorophore, probe, and/or composition of the present invention may be for use in imaging, diagnosing, and/or guidance of treatment of a thyroid disorder.

### Methods

Additional aspects of the invention relate to methods of using the compounds of the invention for imaging or therapy.

One aspect of the invention relates to a method of carrying out a PET scan on a subject, comprising administering to the subject a ligand, fluorophore, probe, and/or composition of the present invention.

Another aspect of the invention relates to a method of imaging tissue comprising a calcium sensing receptor (CSR) in a subject, comprising administering to the subject a ligand, fluorophore, probe, and/or composition of the present invention.

Another aspect of the invention relates to a method of imaging thyroid and/or parathyroid tissue in a subject, comprising administering to the subject a ligand, fluorophore, probe, and/or composition of the present invention.

Another aspect of the invention relates to a method of simultaneously carrying out a PET scan and fluorescence imaging (e.g., NIRF imaging) on a subject, comprising administering to the subject a ligand, fluorophore, probe, and/or composition of the present invention.

Another aspect of the invention relates to a method of identifying parathyroid tissue in a subject, comprising carrying out a PET scan and/or fluorescence imaging (e.g., NIRF imaging) on the subject using a ligand, fluorophore, probe, or composition of the present invention, wherein the PET scan and/or fluorescence imaging identifies presence of parathyroid tissue.

Another aspect of the invention relates to a method of removing hyperplastic and/or ectopic parathyroid tissue in a subject, comprising: (a) carrying out a PET scan and/or fluorescence imaging (e.g., NIRF imaging) on the subject using a ligand, fluorophore, probe, and/or composition of the present invention, wherein the PET scan and/or fluorescence imaging identifies presence of parathyroid tissue; (b) identifying present parathyroid tissue that is hyperplastic and/or ectopic; and (C) surgically excising the identified hyperplastic and/or ectopic parathyroid tissue, thereby removing the hyperplastic and/or ectopic parathyroid tissue.

Another aspect of the present invention provides a method of guiding surgery for the removal of parathyroid tissue in a subject, comprising carrying out a PET scan and/or fluorescence imaging (e.g., NIRF imaging) on the subject using a ligand, fluorophore, probe, and/or composition of the present invention, wherein the PET scan and/or fluorescence imaging identifies presence of parathyroid tissue.

Another aspect of the invention relates to a method of guiding surgery for the protection of parathyroid tissue during thyroid and/or other neck surgery in a subject, comprising carrying out a PET scan and/or fluorescence imaging (e.g., NIRF imaging) on the subject using a ligand, fluorophore, probe, and/or composition of the present invention, wherein the PET scan and/or fluorescence imaging identifies presence of parathyroid tissue.

An additional aspect of the invention provides a method of determining target regions for surgical removal of parathyroid tissue of a subject, e.g., a subject with hyperparathyroidism or a subject at risk for or suspected to have or develop hyperparathyroidism, comprising (a) carrying out a PET scan and/or fluorescence imaging (e.g., NIRF imaging) on the subject using a ligand, fluorophore, probe, and/or composition of the present invention, wherein the PET scan and/or fluorescence imaging identifies presence of parathyroid tissue; and (b) identifying one or more regions(s) of the subject comprising the presence of ectopic and/or hyperplastic parathyroid tissue, wherein the presence of ectopic and/or hyperplastic parathyroid tissue in the one or more region(s) indicates the region(s) as a target region of the subject for surgical removal of parathyroid tissue.

Another aspect of the invention relates to a method of treating hyperparathyroidism (e.g., primary, secondary, and/or tertiary hyperthyroidism) in a subject, comprising determining the suitability of a subject with hyperparathyroidism or a subject at risk for or suspected to have or develop hyperparathyroidism to surgical removal of parathyroid tissue by carrying out a PET scan and/or fluorescence imaging (e.g., NIRF imaging) on the subject using a ligand, fluorophore, probe, or composition of the present invention, wherein the PET scan and/or fluorescence imaging identifies presence of parathyroid tissue, and treating the hyperparathyroidism based on the results of the PET scan and/or fluorescence imaging.

An additional aspect of the invention provides a method of treating a disorder of a calcium sensing receptor (CSR)-positive tissue in a subject, comprising determining suitability of a subject with the disorder or a subject at risk for or suspected to have or develop the disorder to treatment thereof by carrying out a PET scan and/or fluorescence imaging (e.g., NIRF imaging) on the subject using a ligand, fluorophore, probe, or composition of the present invention, wherein the PET scan and/or fluorescence imaging identifies presence of CSR-positive tissue, and treating the disorder based on the results of the PET scan and/or fluorescence imaging.

Non-limiting examples of disorders relevant to the ligands, fluorophores, probes, compositions, and methods of the invention include a parathyroid disorder (e.g., primary hyperparathyroidism, secondary hyperparathyroidism, tertiary hyperthyroidism), a thyroid disorder (e.g., thyroid cancer, goiter, thyroid nodules, Graves' disease), a cardiac disorder (e.g., hypertension), a kidney disorder (e.g., nephrocalcinosis, Rickets, proteinuria), a reproductive disorder (e.g., infertility, impaired embryonic or fetal growth), a lactation disorder (e.g., low milk production), a gastrointestinal disorder (e.g., pancreatitis, diabetes, diarrhea, impaired gut secretion), a bone disorder (e.g., osteoporosis), cancer (e.g., colon cancer), a neurological disorder (e.g., Alzheimer's, epilepsy), and/or a lung disorder (e.g., lung hypoplasia, lung hyperplasia).

In some embodiments, the subject may have hyperparathyroidism (e.g., primary, secondary, and/or tertiary hyperthyroidism) or may be a subject at risk for or suspected to have or develop hyperparathyroidism (e.g., primary, secondary, and/or tertiary hyperthyroidism). For example, in some embodiments, the subject may have primary hyperparathyroidism or may be a subject at risk for or suspected to have or develop primary hyperparathyroidism. In some embodiments, the subject may have secondary hyperparathyroidism or may be a subject at risk for or suspected to have or develop secondary hyperparathyroidism. In some embodiments, the subject may have tertiary hyperparathyroidism or may be a subject at risk for or suspected to have or develop tertiary hyperparathyroidism.

In some embodiments, a subject may have a thyroid disorder or may be a subject at risk for or suspected to have or develop a thyroid disorder, including but not limited to thyroid cancer, goiter, thyroid nodules, and/or Graves' disease.

In some embodiments, the subject may be a pre-operative subject.

In some embodiments, the subject may be an intra-operative subject (e.g., wherein the subject is undergoing surgery (e.g., explorative surgery).

In some embodiments, the identified parathyroid tissue may be ectopic parathyroid tissue and/or hyperplastic parathyroid tissue. In some embodiments, the identified parathyroid tissue may be healthy and/or normal (i.e., non-malignant) parathyroid tissue.

In some embodiments, a method of the present invention may further comprise quantifying size of identified parathyroid tissue in the subject, wherein larger than normal (e.g., 5%, 10%, 20%, 30%, 40% <50%, 60%, 70%, 80%, 90%, 100% larger or more, e.g., 1-fold, 2-fold, 3-fold, 4-fold, 5-fold, 6-fold, 7-fold, 8-fold, 9-fold, 10-fold larger or more, e.g., as compared to a control) identified parathyroid tissue identifies abnormal (e.g., malignant, ectopic, hyperplastic, and/or adenomatous) parathyroid tissue. Quantification of parathyroid tissue size may be performed via any known method in the art, such as but not limited to, via quantification of PET and/or dual tracer probe radioactive label accumulation in the tissue (e.g., as an indirect estimate of size), via visual intensity, and/or via computational methods (e.g., based on results imaging modalities such as but not limited to PET, computer tomography (CT), and/or fluorescence imaging).

In some embodiments, a method of the invention may further comprise excising at least some portion of the identified malignant parathyroid tissue. In some embodiments, a method of the present invention may further comprise excising (all of) the malignant parathyroid tissue, i.e., the whole of the identified malignant parathyroid tissue. In some embodiments, a method of the present invention may further comprise protecting at least some portion of the identified healthy parathyroid tissue from excision during parathyroid surgery, thyroid surgery, and/or other neck surgery. n some embodiments, a method of the present invention may further comprise protecting (all of) the identified healthy parathyroid tissue from excision during parathyroid surgery, thyroid surgery, and/or other neck surgery. i.e., the whole of the identified healthy parathyroid tissue.

In some embodiments, a method of the present invention may further comprise scanning excised thyroid tissue for presence of parathyroid tissue.

In some embodiments of the methods of the invention, carrying out a PET scan on the subject using the ligand, fluorophore, probe, or composition of the present invention may comprise administering about 1 to about 15 mCi of the ligand, fluorophore, probe, and/or composition, e.g., about 1, 2, 3, 4, 5, 6, 7, 8, 9, 10, 11, 12, 13, 14, or 15 mCi or any value or range therein of the ligand, fluorophore, probe, and/or composition.

Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or brain). Administration can also be to a tumor (e.g., in or a near a tumor or a lymph node). In some embodiments, the administering may be via intravenous injection. The most suitable route in any given case will depend on the nature and severity of the condition being imaged and/or treated and on the nature of the particular composition that is being administered.

In some embodiments of the methods of the invention, the labeled CSR ligands and/or fluorophores of the present invention may have a serum stability of at least 70% or higher (e.g., at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or higher, or any value or range therein), for at least 30 minutes or more (e.g., in vivo following administration). For example, in some embodiments, a labeled CSR ligand (e.g., a radioisotope-labeled CSR ligand), and/or a halogenated fluorophore of the present invention may have serum stability of at least 70%, at least 85%, or at least 90%, for at least 30 minutes, at least 60 minutes, at least 90 minutes, at least 2 hours, or at least 3 hours for more following administration.

In some embodiments of the methods of the invention, the labeled CSR ligands and/or fluorophores of the present invention may a metabolic stability of at least 50% or higher (e.g., at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or higher, or any value or range therein), for at least 30 minutes or more (e.g., in vivo following administration). For example, in some embodiments, a labeled CSR ligand (e.g., a radioisotope-labeled CSR ligand), and/or a halogenated fluorophore of the present invention may have a metabolic stability of at least 50%, at least 65%, at least 70%, at least 80%, at least 85%, or at least 90%, for at least 30 minutes, at least 60 minutes, at least 90 minutes, at least 2 hours, or at least 3 hours or more following administration.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

The foregoing is illustrative of the present invention, and is not to be construed as limiting thereof. The invention is defined by the following claims, with equivalents of the claims to be included therein.

### Example 1: Development of Novel PET Agents Targeting CSR Based on Calcimetic Drug and Peptides

This study uses arene C—H radiolabeling approaches (Chen et al. 2019 Science 364:1170-1174) to convert a broad spectrum of organic drugs into PET molecular probes under mild and rapid conditions in a relatively straightforward way. Several novel aromatic ¹⁸F-based PET tracers based on NIRF dyes and drug molecules containing arenes are described herein, including successfully generated ¹⁸F labeled cinacalcet (a small molecule targeting calcium sensing receptor (CSR)) for parathyroid detection, and ¹⁸F labeled NIRF dyes for specific parathyroid and thyroid gland targeting.

Most imaging techniques (ultrasound, CT, MRI, etc.) are structurally based, i.e., they can show the shapes of internal structures. Identification of the structures requires the radiologist's knowledge of anatomy. In many cases, a structure can be identified, but its identity remains unknown. Techniques that target molecules or function specific for a given tissue can be very helpful in identifying an unknown mass. A marker that targets a tissue-specific molecule can provide information regarding the identity of the tissue, in a way that structural imaging cannot. For example, a small nodule may be seen near the thyroid on ultrasound, but its identity as a parathyroid adenoma as opposed to a lymph node or focus of ectopic thyroid tissue may not be readily apparent. A parathyroid-specific tracer that “lights up” the nodule could clarify the identity of the nodule. Such an approach would be complementary to structural imaging, and would likely be used in concert with other imaging techniques. Indeed, normal parathyroid are 3-5 mm tissues that are difficult to detect using traditional methods. Once the gland(s) becomes hyperparathyroidism, its size grows to around 10 mm or larger. Unfortunately, current imaging may identify multiple potential lesions (some lesions may not relate to parathyroid which could lead to unnecessary surgery), which makes it important to determine which lesion is related to parathyroid (for surgery planning). Moreover, current imaging is not good at detecting small lesions.

In the present study, several example PET probes and PET/NIRF probes were developed based on CSR binding molecules.

Existing archival clinical specimens were used to test for CSR abundance using immunohistochemistry (IHC). Normal parathyroid glands are occasionally removed inadvertently during thyroidectomy. Existing clinical specimens were reviewed to determine suitable tissue blocks. Using standard IHC methods, tissue was stained for expression of CSR. Expression was scored using the “H” score, which takes into account the intensity of staining (0-3) and the proportion of cells that stain at each intensity. For example, if 30% of the cells stain 1+, 20% stain 2+and 40% stain 3+the H score would be 30+40+120=190. The range of an H score is 0-300. In addition, parathyroid adenomas, and hyperplastic parathyroids were found among existing clinical specimens. They were also stained for CSR and compared to normal parathyroid glands. 30 normal parathyroid glands with adjacent thyroid, 30 parathyroid adenomas, 30 hyperplastic parathyroid glands from patients with primary hyperparathyroidism, and 30 glands from patients with secondary (renal) hyperparathyroidism were used in this study. Patients with hyperplasia were identified based on review of the entire clinical record, as routine histology cannot reliably distinguish between an adenoma and hyperplasia. A difference in staining of 3-4 fold was anticipated be adequate to distinguish the tissue on imaging.

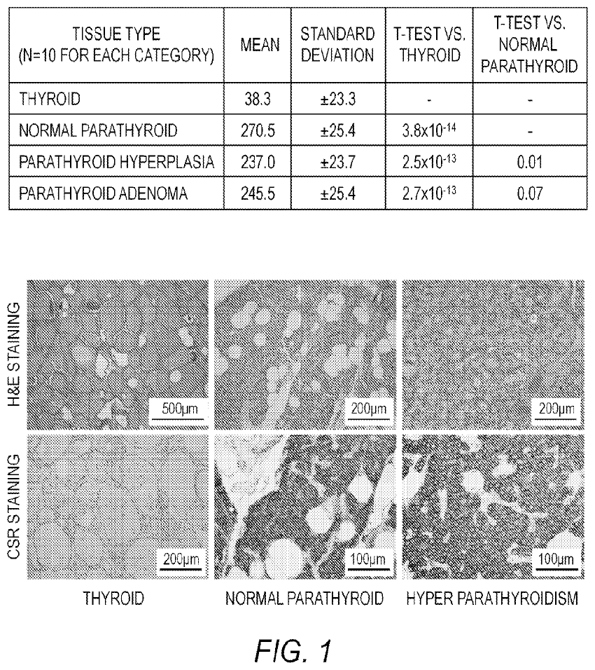

FIG. 1 shows the results of CSR staining on normal thyroid, parathyroid, and hyper-parathyroidism, and demonstrates that CSR expression in hyperparathyroidism and parathyroid glands is 6-7 times higher than its expression in nearby thyroid tissue. This difference provides adequate contrast in PET imaging, and high contrast was observed in following rat and NHP studies. These staining results clearly demonstrated CSR as a valid target for parathyroid imaging.

Cinacalcet was used a starting example to develop novel parathyroid targeted PET agents based on calcimimetic drugs. Cinacalcet is a therapeutic drug that binds the CSR with high affinity and selectivity. In order to successfully convert cinacalcet into an imaging agent, the hydrogen on the cinacalcet aromatic ring was replaced with fluorine, which represents a minimal change on the chemical structure, aimed to maintain CSR binding affinity of the parent compound (FIG. 2, top schematic “A”). Traditionally, it has not been straightforward to introduce ¹⁸F into an electron-rich aromatic system. Recent work by the inventors of the present invention on photoredox radiolabeling provided an innovative method to convert a C—H bond to a C—F bond directly. By replacing the H with a similar sized F the aim was to maintain the binding affinity towards CSR, which was confirmed by binding assay. Synthesis of the optically pure cinacalcet was followed by one step Boc protection. Other ¹⁸F labeled cinacalcet analogs were also prepared using similar strategy. In addition to direct C—H fluorination, radiofluorination of CF₃ group in cinacalcet was also explored (FIG. 2, bottom schematic “B”). Through Frustrated Lewis Pair mediated method, Boc protected cinacalcet was prepared through either direct radiofluorination of FLP or a relatively more stable Br intermediate. After one step deprotection, the radioactive molecule bearing the same chemical structure of parent cinacalcet drug was obtained. After the precursors were obtained, radiolabeling conditions were explored by varying light source, temperature, fluoride source, reaction ratio etc. Selected agent had an isolation yield of >5%.

Fluorine-18 (¹⁸F) is one of the most important radioisotopes in the radio-pharmaceutical industry, as it possesses a relatively long half-life (t_{1/2}=110 min) and decays with high efficiency by positron emission (97%). Aromatic or heteroaromatic systems are commonly seen in small molecule pharmaceuticals and therapeutics, making the direct conversion of arene C—H into C—¹⁸F bonds ideal due to the prevalence of aromatic C—H bonds and the increasing importance of C(sp²)-F bonds in small molecule therapeutics and probes. A previous study on direct C—H [¹⁸F] fluorination of aromatics that makes use of [¹⁸F]TBAF as the source of [¹⁸F] fluoride published a list of model compounds (Chen et al. 2019 Science 364:1170-1174; incorporated herein by reference). In addition to this report, a deoxy-radiofluorination was recently discovered which allows site specific introduction of ¹⁸F to aromatic systems using blue laser at ice-cooled temperature (Tay et al. 2020 Nature Catalysis. 2020. 3:734-742; incorporated herein by reference).

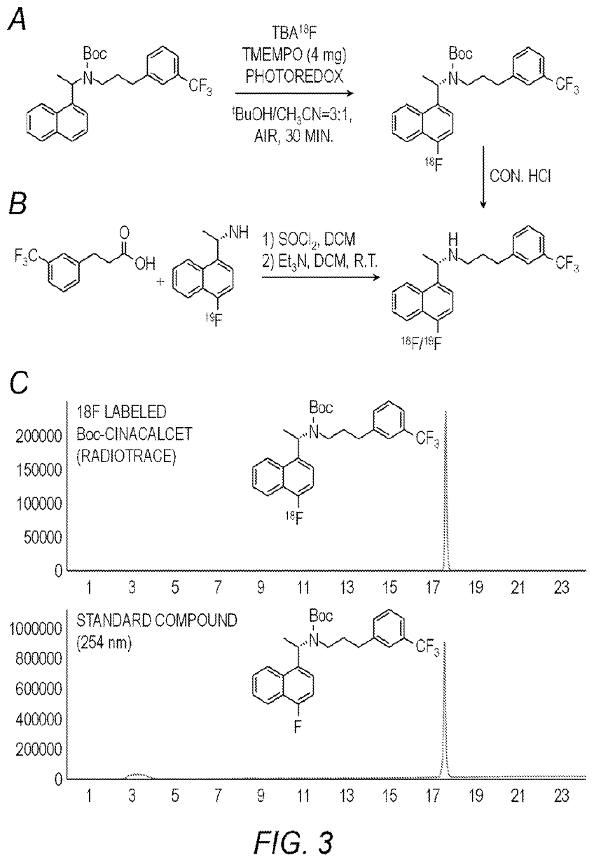

In this study, direct [¹⁸F] radiofluorination of arene C—H bond of Boc protected cinacalcet was performed (FIG. 3). Photoredox labeling generated the desired product with 19% isolation yield. The ¹⁸F labeled Boc-cinacalcet could be completely separated from Boc-cinacalcet using HPLC separation. The labeling primarily occurred at position 4 of the naphthalene ring and the identity was confirmed by co-injection with the 4F-Boc-cinacalect standard. There were smaller radiopeaks seen that may be compounds labeled at other positions, all of which could be completely separated from 4-[¹⁸F]F-Boc-cinacalcet. After removing the Boc group, 4-[¹⁸F]F-cinacalcet could be obtained with >98% radiochemical purity and 2.1 Ci/μmol molar activity, which was then injected to a normal animal for initial evaluation.

After obtaining the desired agents targeting CSR, their dissociation constant (Kd) was determined using saturation assay. In brief, O cells (positive CSR expression) were seeded in two 24-well plates with 0.2×10⁶ cells/well for incubation overnight at 37° C., 5% CO₂. Then stock solution of the radiotracer in FBS-free medium was prepared at concentration of 0.1 nM to 100 uM range through adding cold standard. The increasing concentration of radiotracer solutions was successively added into associated wells in one plate for measuring cell-bound binding, and nonspecific binding of tracer was assessed in another plate in the presence of a large excess of non-radiolabeled compound (500 μM). After incubation for 1 h on ice, unbound tracer was removed and washed softly three times with ice cold PBS, and cells were harvested with 0.2 N NaOH for measuring radioactivity with gamma counter. Specific binding was determined by subtracting nonspecific binding from cell-bound activity, and Kd value was calculated through specific binding curves by using nonlinear regression curve fits (GraphPad Prism). Desired agents should have comparable Kd compared with cinacalcet and etelcalcetide. In addition to Kd value, serum stability and metabolic stability were also performed. The selected agents should have serum stability of >90% and metabolic stability >80% at 1h post incubation/injection.

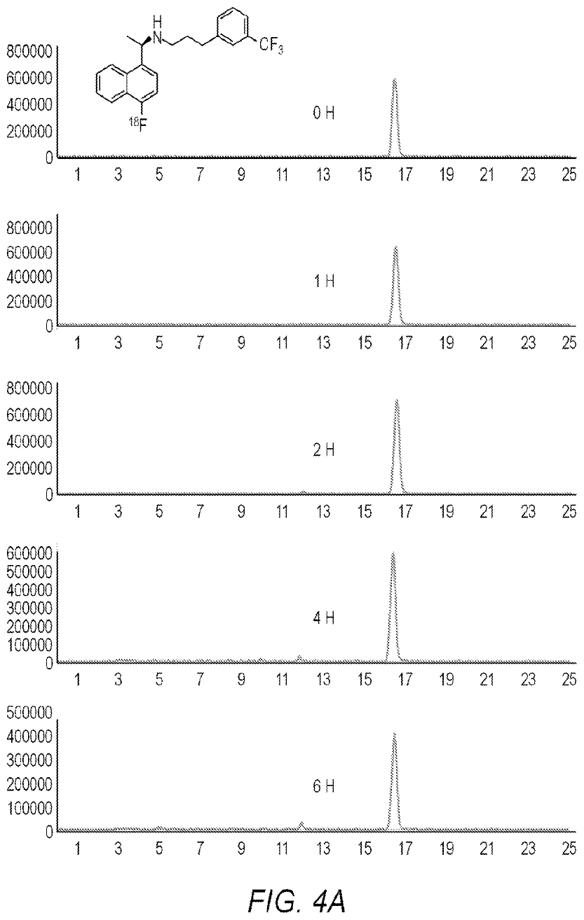

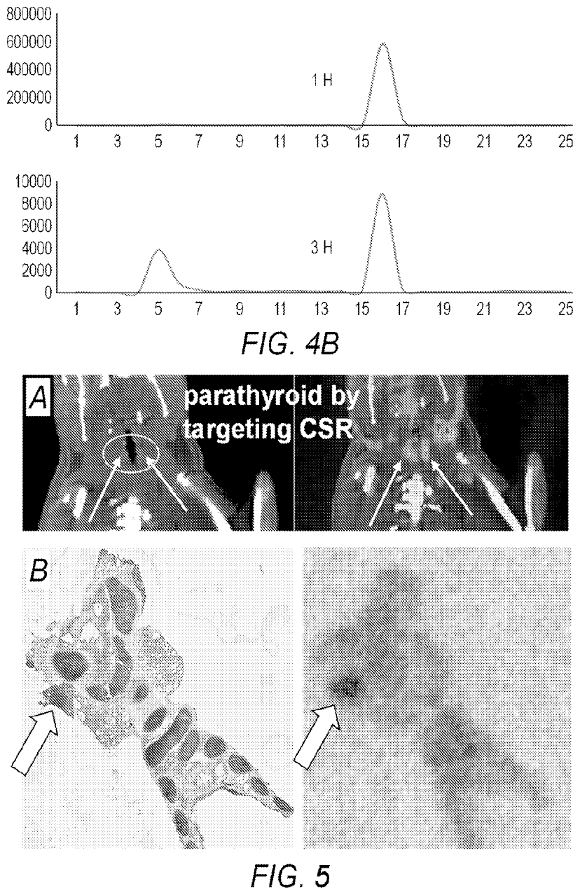

In vitro stability of ¹⁸F-cinacalcet was performed, which demonstrated >90% purity at 6 h time point (FIG. 4A). In a non-human primate imaging experiment, the blood samples were drawn from vein at 1 and 3 h post injection. As shown in FIG. 4B, the agent is rather stable at 1 h time point and significant metabolites are observed at 3 h time point. Because a PET imaging would be finished at 1 h post injection, the obtained results suggest ¹⁸F-cinacalect has reasonable stability as a PET agent.

Normal rats were used as an animal model for initial CSR agent imaging. Surgical dissection of the mouse parathyroid gland is too technically difficult, in contrast to the rat which has approximately 10 times the body mass of a mouse. Analyses were performed by individuals with extensive experience on parathyroid identification for pathology. autoradiography, and microscope study. In brief, animals were anesthetized with isoflurane. For PET probes, dynamic PET imaging was performed after the administration of 1 to 2 mCi of tracer, and animals were imaged with 2 h dynamic scan. A CT scan was also obtained for anatomic registration and attenuation correction. Images were reconstructed to create representative dynamic images for the study (10 min epochs). These images were qualitatively graded to assess the visibility of the parathyroid gland against background tissues. PET kinetic modeling was also performed to evaluate the relative kinetics of uptake of the parathyroid gland compared to the thyroid and other tissues.

After the imaging of each animal, the parathyroid glands were carefully dissected out and the uptake in parathyroid gland was measured, then subsequently evaluated pathologically to confirm the histology. Autoradiography was performed to study its distribution in gland area. Optical imaging guidance during the surgical procedures was also performed using the developed CSR PET-fluorescence probes. During excision, the surgeon also graded the visibility of the lesions with and without the use of fluorescence. Other critical organs, including the thyroid gland, nearby tissues, and major organs were also dissected out to evaluate the relative uptake within these organs. The localization of radioactivity in the gland tissue was also confirmed by autoradiography coupled with pathology staining of neighboring slides.

¹⁸F-cinacalcet could detect parathyroid gland in rats by targeting CSR. Parathyroid gland are very small (3-5 mm in human) and generally below the detection limit of most imaging methods. The novel CSR PET agent ¹⁸F-cinacalcet was injected in rat and small animal PET imaging was performed. As shown in FIG. 5 panel A, the parathyroid region of rat neck area clearly demonstrated two hot spots. In order to confirm the observed signal did come from the very small parathyroid gland instead of thyroid or other tissue, the rats were sacrificed and the localization of radioactivity in the gland tissue was evaluated by autoradiography coupled with pathology staining of neighboring slides. As shown in FIG. 5 panel B, localization of ¹⁸F-cinacalcet correlated well with parathyroid gland which is CSR positive. These results strongly supported the approach of using CSR for parathyroid imaging.

In addition to cinacalcet, etelcalcetide is another calcimimetic drug for the treatment of secondary hyperparathyroidism. This peptide could also be modified to construct PET and PET-fluorescent probes.

### Example 2: Development of ¹⁸F Labeled NIRF Dyes Targeting Parathyroid Gland

Previously, novel halogenated fluorophores have been discovered for thyroid (T700) and parathyroid (T800) fluorescent imaging without any targeting motif (Wizenty et al. 2020 Molecules 25; Kim et al. 2017 Gland Surg. 6:516-524; Wada et al. 2017 Ann. Thorac. Surg. 103:1132-1141; Hyun et al. 2015 Nat. Med. 21:192-197). Real-time, high sensitivity NIRF imaging allows the distinction of parathyroid from thyroid and surrounding soft tissue. Clear visualization of parathyroid glands would aid in parathyroid surgery and also minimize thyroid injury or removal normal tissues. However, NIR fluorophores still have limited tissue penetration. This could be especially problematic in detecting parathyroid glands at ectopic locations, or when in hidden locations such as within the thyroid. In order to solve this limitation, this study replaced the ¹⁹F element within the NIRF dye with PET isotope ¹⁸F, allowing the agent to be detected by PET for parathyroid location while still maintaining the NIRF property for image-guided surgery. Because the radioactive compounds have the exact same structure of the parent dye, the parathyroid or thyroid homing capability of the dyes would not be changed. The data herein describe successful generation of ¹⁸F labeled T700 and T800 which demonstrated preferential uptake in thyroid and parathyroid glands, respectively. These radioactive and fluorescent agents could be used to detect parathyroid with PET followed by image-guided surgery.

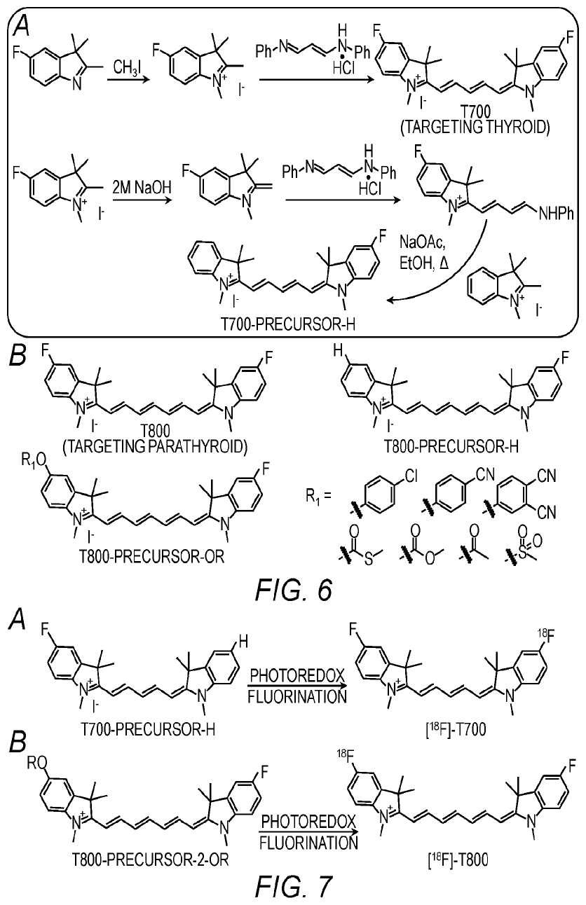

The optical signal of halogenated cyanine dyes T700 (taken up in thyroid) and T800(taken up in parathyroid) only have limited tissue penetration. The standard compounds and asymmetric precursors of T700 and T800 were prepared as shown in FIG. 6. Starting from asymmetric precursors T700-precursor-H and T800-precursor-H, direct C—H radiofluorination leads to the symmetric products ¹⁸F-T700 and ¹⁸F-T800 (FIG. 7). Because C—H fluorination of the T700-precursor-H and T800-precursor-H may radiofluorinate several arene C—H sites, ¹⁸F-T700 and ¹⁸F-T800 were also synthesized through the newly developed deoxyradiofluorination method (Tay et al. 2020 Nature Catalysis 3:734-742). In brief, the asymmetric precursor T800-precursor-OR was prepared as shown in FIG. 6. Radiofluorination took place at the position bearing “OR” groups. The nucleofuge scope for deoxyradiofluorination includes 4-chlorophenoxy ethers and other electron-poor aryloxy groups as shown in FIG. 6. Selected ¹⁸F-T800 for further use need to maintain a quantum yield>5% with an excitation>700 nm emission>720 nm (ensure labeling process do not negatively impact its optical signal) and serum stability >90% at 1 h incubation for in vivo study

[¹⁸F]-radiolabeling was performed through the formation of stable C—[¹⁸F]F bond, using either direct C—H fluorination or the deoxyfluorination method according to the general labeling scheme shown in FIG. 7. In vitro stability of these novel PET/fluorescence dyes was evaluated by HPLC at different time points (0.5 h, 1 h, 2 h, 4 h, and 6 h) after incubation in PBS and bovine serum albumin (BSA). Their resistance (or sensitivity) to radiolysis, especially at high activity concentration, was also investigated. The information was combined with obtained fluorescence property of these fluorinated dyes as described above (excitation/emission wavelength, quantum yield, and photostability) to determine if the [¹⁸F]-T700/T800 dyes demonstrated reasonable stability in vitro (>90% at 1 h incubation, no defluorination). Selected agents meeting these criteria were injected into normal mice for in vivo stability evaluation.

Currently, it is still unknown why T800 and T700 are taken up by different glands. However, the halogenation and the length of polymethine appears to be important. While not wishing to be bound to theory, it was hypothesized that thyroid uptake of T700 may be a result of an active transport mechanism mediated by the NIS protein. A blocking study in the presence of I⁻ would therefore evaluate whether its uptake could be reduced. Similarly, T800 may be a calcimimetic, and a blocking study in the presence of Calcium ion would evaluate the blocking effect.

To confirm the localization profile of T700 and T800 dyes and evaluate the newly made T800 analogs, in vivo tests were performed on normal rats. Considering the small size of normal parathyroid in rats, fluorescent microscope and pathology were used to confirm parathyroid uptake and contrast after PET/NIFR imaging. In brief, the imaging agents were injected i.v. and imaged with IVIS and PET/CT after 1 and 4 h post injection. To determine the dose of the imaging agent, a range of 2 nmol to 100 nmol of imaging agent were injected to select the optimal dose that has high parathyroid signal without inducing too much background signals. After the imaging experiment was finished, the parathyroid gland was removed for further characterization on signal to noise contrast with fluorescence microscopy, autoradiography (neighboring slides), and pathology. Signal-to-background ratio was calculated using fluorescence intensities and/or autoradiography signal between parathyroid and thyroid or neighboring tissues. The selected agent should have signal-to-background ratio >2.

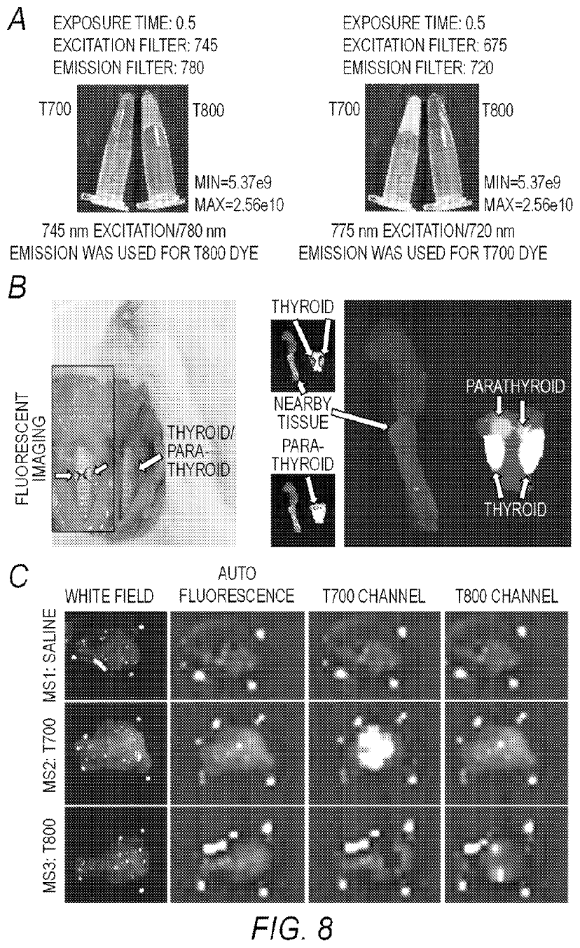

Both T700 and T800 were synthesized and then scanned in IVIS using dual channel imaging. As shown in FIG. 8 panel A, the 675 nm excitation 720 nm emission channel leads to the predominant fluorescent signal from T700. In contrast, the 745 nm excitation 780 nm emission channel leads to the predominant fluorescent signal from T800. Clearly, these parameters could be used in our dual channel imaging. To confirm the gland specific imaging capability of T700 and T800, 0.2 μmol of each of the agents was injected to Wistar rats and ex vivo imaging was then performed. As shown in FIG. 8 panel B, a prominent fluorescent signal was observed in parathyroid from the T700 signal. Ex vivo scan further confirmed the T700 signal is located at thyroid gland and the T800 signal is located in parathyroid location. Clearly, dual channel overlay would help surgeon to identify parathyroid glands with respect to the background thyroid and nearby tissues.

Although rat parathyroid is much larger than mouse parathyroid, ex vivo imaging may still lack the needed resolution due to the overall small size of the gland. The ability to accurately identify parathyroid glands is an important technique in order to evaluate parathyroid uptake and parathyroid to background contrast through fluorescent microscope or autoradiography in initial screening studies. Autofluorescence from parathyroid was also examined (FIG. 8 panel C). Mouse 1, 2 and 3 was injected with saline, T700 and T800 dye, respectively. The tissues containing parathyroid was imaged at autofluorescence, T700 and T800 channel. As expected, the autofluorescence was at almost background level in parathyroid location.

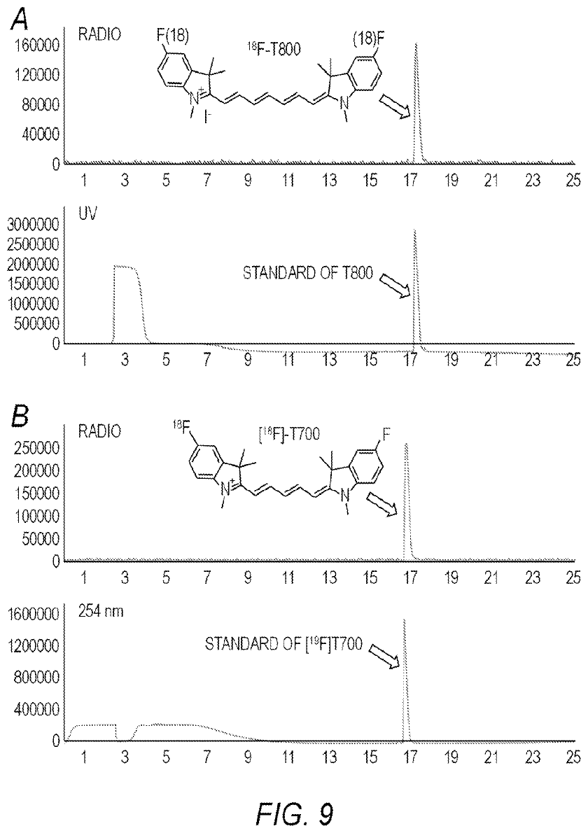

The synthesized [¹⁸F]-T800 and [¹⁸F]-T700 were confirmed with radio-HPLC to correlate well with the standards (FIG. 9). The traditional labeling method using nitro as leaving group failed to lead to the desired product. An important step of the methodology was introducing ¹⁸F to the aromatic ring of cyanine dyes (T700 and T800). This initial success reduces confirms the feasibility of the methods proposed herein. Other labeling methods could include radio-deoxyfluorination (Neumann et al. 2016 Nature 538:274), fluorination of N-arylsydnone (Narayanam et al. 017 Angew Chem. Int. Ed. Engl. 56:1306-13010), sulfonium (Gendron et al 2018 J. Am. Chem. Soc. 140:11125-11132) and iodonium salts (Ichiishi et al. 2014 Org. Lett. 16:3224-3227; McCammant et al. 2017 Org. Lett. 19:3939-3942; Rotstein et al. 2014 Nat. Commun. 5:4365), fluorodemetalation (Lee et al. 2011 Science 334:639-642; Lee et al. 2012 J. Am. Chem. Soc. 134:17456-17458), and copper-mediated cross-coupling of boronic acids (Mossine et al. 2015 Org. Lett. 17:5780-5783) and esters (Tredwell et al. 2014 Angew Chem. Int. Ed. Engl. 53:7751-7755). There may be concerns that the hydrophobicity of the dyes would lead to high background signal. Data herein demonstrated good contrast of T800 in parathyroid compared with nearby tissues. For photoredox radiofluorination, there may multiple potential-reaction sites and the ability to be separated from the precursor. The reaction may have two or sometimes three major products for CH fluorination. S_{N}Ar photoredox deoxyradiofluorination would mainly lead to one product. The labeled agent can be well separated on an F5 column from the precursor. Lead agents were further evaluated in nude mice bearing transplanted human parathyroid tissue. These experiments ensured that the developed agents could target human parathyroid tissue efficiently in addition to rodent parathyroid.

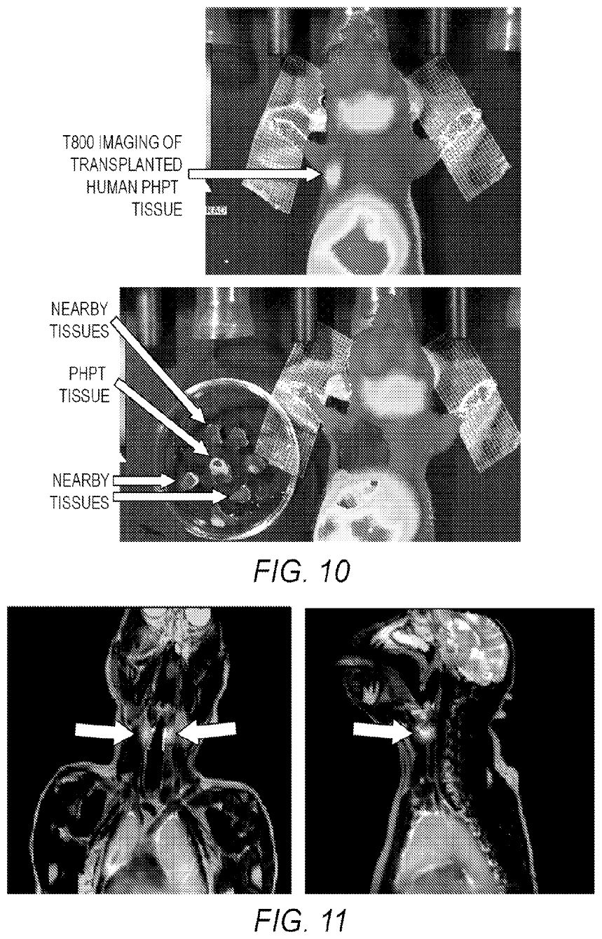

In order to evaluate newly developed agents in a clinically relevant model, xenografting of human parathyroid tissue was used. Over 15 mouse models were established by transplanting human parathyroid glands into nude mice. Using the established T800 as the probe, the transplanted PHPT tissue could clearly be visualized after the blood vessels had been established at the implantation site (FIG. 10). The transplants were also surgically removed along with nearby tissues. The ex vivo imaging demonstrated good contrast between parathyroid and nearby tissue.

Further experimentation includes performance of a 14-day toxicology study in rodents. A single high dose (>100-fold higher than the anticipated human dose for the selected agent) is used for the acute toxicity study. The study is performed under good laboratory practices (GLP) conditions. Male and female rats are administered a single i.v. dose of the lead agent (>100 fold imaging dose). The animals are checked twice per day for morbidity/mortality and signs of toxicity. Animals have bodyweights, blood chemistry and hands on observations data collected before injection, and at day 1, 2, 4, 8, and 14 Day. Four groups of animals are tested. Goup1: no fasting control; group 2: no fasting+lead agent; group 3: 12 h fasting; and group 4: 12 h fasting followed by injection of the lead agent. On day 15, all remaining animals are euthanized followed by blood and urine collection and analysis, clinical chemistry, hematology, and coagulation factors. Major organs are collected, weighted, and processed for H&E staining and examination. Statistical analysis among different groups is conducted on the aspects of body weight, organ weight, clinical pathology, and urinalysis data, etc.

### Example 3: Lead Agent Use in Non-Human Primates

One or more lead agent is selected for further characterization in nonhuman primates, e.g., rhesus macaque.

After injection with ¹⁸F-labeled agent i.v., a 2-hour dynamic scan is performed. A static scan is also performed centering at the thyroid and parathyroid region. The goals of this experiment are to (1) characterize the pharmacokinetics, biodistribution, and metabolic stability of ¹⁸F-labeled PET agent during the first 2 h following brief i.v. infusion; (2) estimate dosimetry data from future proposed clinical procedure; (3) design a clinical PET/CT scan protocol, including injection dose and scan time points, from which an optimal time for maximum imaging quality and parathyroid gland-to-background contrast will be determined.

In detail, Rhesus Macaque (NHP) are maintained on 1.4-4% isoflurane inhalation anesthesia and artificial ventilation. Two venous catheters are applied, one for tracer administration and one for sampling of blood radioactivity concentration. A CT transmission scan is obtained. Then, ¹⁸F-labeled PET agent targeting parathyroid (3-5 mCi) is given i.v. and a 120-min dynamic PET scan is performed. The scans are performed with and without fasting to compare the uptake and contrast difference. Serial venous blood samples (0.2-0.5 ml) are drawn before and at 0.5, 5, 30, 60, and 90 min p.i. to determine metabolic stability and blood uptake. Body temperature, heart rate, ECG, pCO₂, pO₂, SaO₂ and blood pressure are monitored throughout the study. A urine specimen for HPLC metabolite analysis is collected at the end of the whole body scan. PET scan data is analyzed, including volumetric region of interest (ROI) analysis and extraction of tissue TACs and steady-state SUVs; quantitative analysis of plasma time-activity-curves (TACs) and HPLC data to determine TACs for circulating ¹⁸F-PET agent and its metabolites vs. time; and calculated cumulated activities for normal organs/tissues.

Blood plasma and urine samples are assayed for ¹⁸F-labeled agent and labeled metabolites. The blood samples are collected and immediately centrifuged for 5 min at 14,000 rpm. Then, 50% TFA in 100 μL of PBS is added to the upper serum solution, followed by centrifugation for 5 min. The upper solution is injected for HPLC analysis. Urine is filtered, and then used for HPLC analysis.

The distribution of absorbed dose is calculated according to the MIRD method, which assumes that the integrated activity is known for each of the source organs. Observed source organs where ¹⁸F-PET agent may be concentrated include the urinary bladder, kidneys, and liver. Other organs for which anatomic boundaries can be identified using a combination of the ¹⁸F-PET scan, the attenuation scan, and the comparison CT scan are used as additional source organs for completion (brain, lower large intestine, stomach, blood, heart wall, lung, pancreas, red marrow, spleen). Organs within which no ¹⁸F-PET uptake above background is observed and for which boundaries cannot be delineated are treated as background and assigned the remainder level of cumulated activity.

NHPs will be closely monitored for the development of toxicity. Metabolic studies are performed, including blood chemistry profile (electrolytes, glucose, calcium, phosphorous, magnesium, bilirubin, albumin, total proteins, AST, ALT, ALP) to assess potential liver and kidney function changes.

Primary hyperparathyroidism is more common in women with an incidence of 66 per 100,000 person-years in women and 25 per 100,000 person-years in men. Accordingly, both male and female subjects are used for the preclinical biodistribution and imaging studies described herein. The potential difference between genders is compared and calculated in statistical analysis.

Native parathyroid PET imaging in Rhesus Macaque was performed with the newly developed ¹⁸F-cinacalcet. Importantly, this CSR agent demonstrated prominent uptake in parathyroid region as shown in FIG. 11, indicating CSR is a valid target for parathyroid imaging. Blood metabolic stability indicated ¹⁸F-cinacalcet has acceptable stability for imaging applications (FIG. 4 panel B).

### Example 4: Development of CSR Targeted PET Agent for Parathyroid Gland Imaging

The introduction of ¹⁸F (radio tag) into BOC-protected Sensipar® (cinacalcet, a small molecule targeting CSR) was performed efficiently using direct photoredox C—H radiofluorination. After simple deprotection, ¹⁸F labeled cinacalcet (named [¹⁸F]F-ZW-cinacalcet) was obtained in 34% yield, whose uptake could be efficiently blocked by competitors. The agent also demonstrated fast blood clearance and good plasma stability. The [¹⁸F]F-ZW-cinacalcet PET imaging noninvasively detected parathyroid glands in mice and rats, whose localization was further confirmed by autoradiograph and immunohistochemistry. To further facilitate future clinical translation, PET/MRI scan in non-human-primates (NHPs) was performed. [¹⁸F]F-ZW-cinacalcet demonstrated apparent tissue accumulation in the parathyroid region. Toxicity studies indicated [¹⁸F]F-ZW-cinacalcet is safe for future human studies. In summary, the data demonstrated [¹⁸F]F-ZW-cinacalcet is a novel imaging agent for parathyroid detection, which would benefit the management of patients with primary hyperparathyroidism.

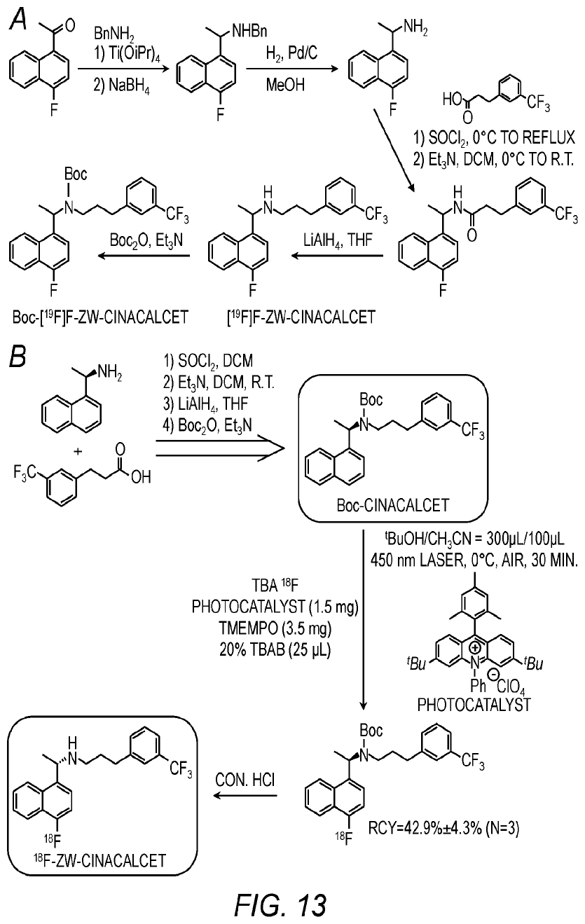

Synthesis of [¹⁹F]F-ZW-cinacalcet and the precursor for the photoredox reaction. The standard compound of F-ZW-cinacalcet was synthesized based on the scheme shown in FIG. 13 panel A. In brief, 4-fluoro-1-acetonaphthone was reduced to N-benzyl-1-(4- fluoronaphthalen-1-yl)ethan-1-amine in two steps with 53% yield. After removing the Bn protecting group, the conjugation with 3-(trifluoromethyl)hydrocinnamic acid was performed (87% yield), followed by amide bond reduction with NaBH₄ (97% yield), which led to the desired standard of F-ZW-cinacalcet. A Boc group was also added to F-ZW-cinacalcet to generate the standard-Boc-F-ZW-cinacalcet. To synthesize a precursor compatible with the photoredox labeling reaction, a Boc protecting group was added to the parent drug cinacalcet (FIG. 13 panel B). Protecting the secondary amine avoided the potential oxidation at the nitrogen atom and favored direct C—H radiofluorination through photoredox reactions.

Photoredox radiofluorination. Cinacalcet is a therapeutic drug that binds the CSR with high affinity and selectivity. Although ¹⁸F could be introduced to the CF₃ group of Cinacalcet (a SP³-¹⁸F bond), the resulting agent has poor stability. There is a need to design a new Cinacalcet based agent to improve the agent stability with a minimal change to the parent drug structure. A photoredox labeling method allowed the formation of C—F bonds through direct C—H radiofluorination. This SP²-F bond in naphthalene was more stable compared to the SP³ bond. The agent only replaced one arene-CH bond with an arene-CF bond, which represented minimal change of the parent drug structure.

The synthesis of [¹⁸F]F-ZW-cinacalcet was performed in two steps: direct C—H radiofluorination under photoredox conditions, followed by deprotection of the Boc group. Starting from azeotropically dried [¹⁸F]F-TBAF, direct [¹⁸F]radiofluorination of the arene C—H bond of Boc protected cinacalcet led to Boc-[¹⁸F]F-ZW-cinacalcet in 42.9±4.3% yield under optimized photoredox labeling conditions (450 nm, 3 W laser, 30 min irradiation). The labeling occurred primarily at position 4 of the naphthalene ring and the identity is confirmed by the co-injection with Boc-[¹⁹F]-ZW-cinacalcet standard. After removing the solvent, concentrated HCl was added followed by heating at 95° C. for 10 min to remove the Boc protecting group (80% yield). The final agent [¹⁸F]F-ZW-cinacalcet was obtained with >96% radiochemical purity and 4.6 Ci/μmol activity. Although an ice bath was used for the labeling reaction, the radiofluorination can also proceed without the cooling setup. The solvent evaporates faster and special attention is needed to avoid complete dryness of the reaction. The reaction can also be scaled up to produce enough agent for non-human primate studies. Due to the hydrophobicity of the agent, 50% EtOH was used to reduce the sterile filter absorption of the agent in the final step.

Stability of [¹⁸F]F-ZW-cinacalcet. The photoredox labeling method allows access to a new category of PET agent bearing a SP²-F bond. [¹⁸F]F-ZW-cinacalcet has improved stability compared to the CF₃ labeling method. The formulated [¹⁸F]F-ZW-cinacalcet was first incubated in PBS (Phosphate buffered saline containing 8% EtOH), and aliquots were taken for analysis at different time points. Although a minor hydrophilic impurity peak was observed at 2 h post incubation, the purity was maintained at >95% at 4 h post incubation. Subsequently, the in vivo stability of [¹⁸F]F-ZW-cinacalcet was tested in a non-human primate. After injection, a blood sample was collected at 1 and 3 h time points. The majority of the agent remained untouched at the 1 h time point. A hydrophilic metabolite was observed at the 3 h time point. Because the imaging was finished within the first hour, the metabolites were not determined in this study

Determination of Octanol water Partition Coefficient (Log P). The Log P of [¹⁸F]F-ZW-cinacalcet was determined in a 1-octanol and water system with an average value Log P=1.95±0.02. The result indicated that [¹⁸F]F-ZW-cinacalcet has lipid solubility.

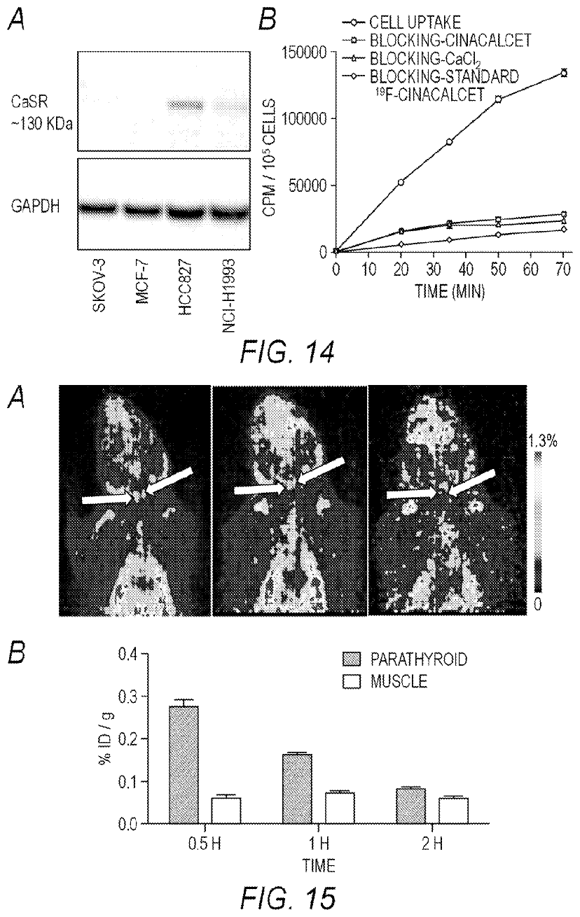

Cell uptake and specific blocking assays. In order to verify the target specificity of [¹⁸F]F-ZW-cinacalcet towards CSR, cell uptake and blocking experiments were performed. As shown in FIG. 14 panel A, Hcc827 (non-small cell lung cancer cell line) has high CSR expression, which was then selected for the in vitro assays. When incubated with [¹⁸F]F-ZW-cinacalcet, the radio-activity uptake by Hcc827 cells gradually increased over time (FIG. 14 panel B). To confirm the binding specificity, Hcc827 cells were co-incubated with [¹⁸F]F-ZW-cinacalcet and excess amount of cinacalcet, CaCl₂ and cold standard [¹⁹F]F-ZW-cinacalcet (100 μM). Among the blocking agents, [¹⁹F]F-ZW-cinacalcet could efficiently block the uptake of [¹⁸F]F-ZW-cinacalcet by 89.9% after 20 min post incubation. More than 87% uptake was observed for the other time points. The uptake of [¹⁸F]F-ZW-cinacalcet was also efficiently blocked by cinacalcet (71.3±2.9%˜79.0±0.6% reduction) and CaCl₂ (71.5±1.2%˜82.9±1.7% reduction). The results indicated that [¹⁸F]F-ZW-cinacalcet can be taken up by CSR expressing Hcc827 and the target specificity was confirmed by competitive blocking studies. [¹⁸F]F-ZW-cinacalcet allowed visualization of CSR expression in vivo through targeted molecular imaging.

Small animal-PET CT imaging of parathyroid in rodents. Because the parathyroid is rather small, rats were used instead of mice to evaluate the agent. Static PET scans were acquired in rats at 0.5 h, 1 h and 2 h post injection of [¹⁸F]F-ZW-cinacalcet. Representative coronal PET images are shown in FIG. 15 panel A. Parathyroid could be visualized on the image at the early time point of 0.5 h with a radiotracer uptake of 0.28±0.16% ID/g and parathyroid/muscle ratio of 4.9±0.93. The parathyroid radioactivity decreased over time and reverted to the background level at the 2 h time point (FIG. 15 panel B). Uptake in the salivary gland was also observed, which did not decrease overtime. Interestingly, a high uptake of radioactivity in the rat's lung was observed, which was confirmed to have high CSR expression by staining.

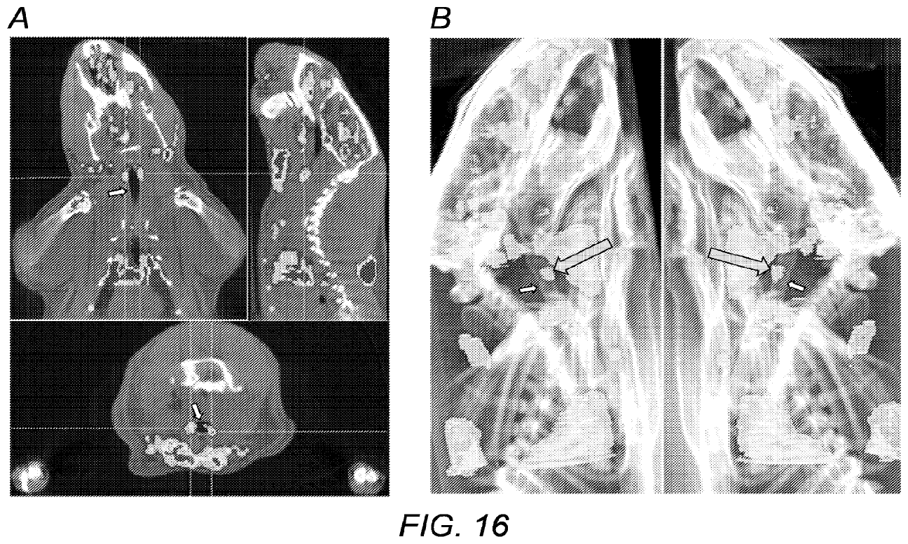

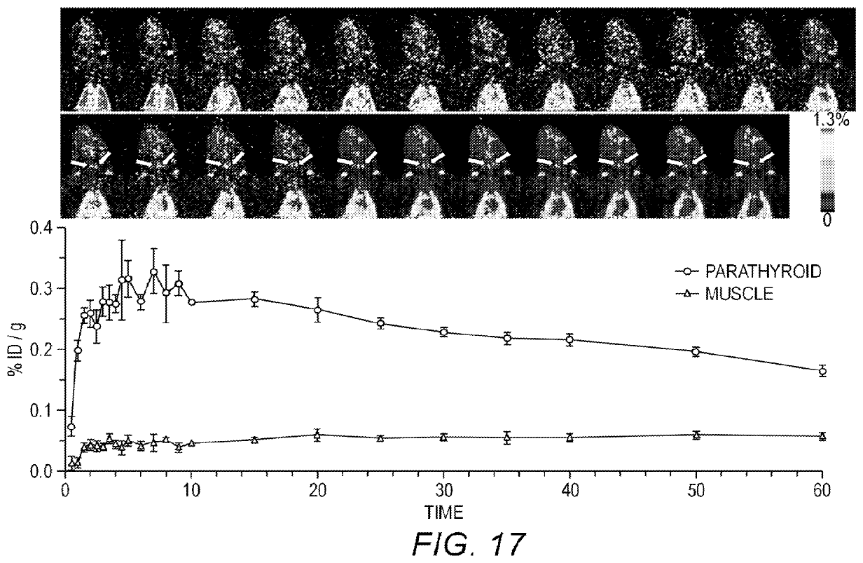

To better illustrate the location of [¹⁸F]F-ZW-cinacalcet in vivo, PET and CT images were merged at coronal, sagittal, and transverse levels (FIG. 16 panel A), respectively, and 3D volume-rendered PET/CT images with multi-angles were also reconstructed (FIG. 16 panel B). From the spatially oriented PET/CT images, it was easy to discern the parathyroid adjacent to the cricoid cartilage and trachea. The radioactive uptake of the parathyroid glands was significantly higher than that of the background tissues.

Dynamic PET images of [¹⁸F]F-ZW-cinacalcet in rats were acquired for 60 minutes. Representative PET images and regional time-radioactivity curves of parathyroids and muscles are shown in FIG. 17. [¹⁸F]F-ZW-cinacalcet accumulated into parathyroid at the earliest timepoints and peaked at about 7 min post injection (0.33±0.05% ID/g), followed by a gradual decline to 0.17±0.01% ID/g at 60 min post injection. Muscle uptake of [¹⁸F]F-ZW-cinacalcet was significantly lower at all timepoints (i.e., <0.06% ID/g±0.01 at 20 min post injection)). These results indicated excellent specificity and tissue kinetics (“fast in” and slow clearance) of [¹⁸F]F-ZW-cinacalcet in parathyroid.

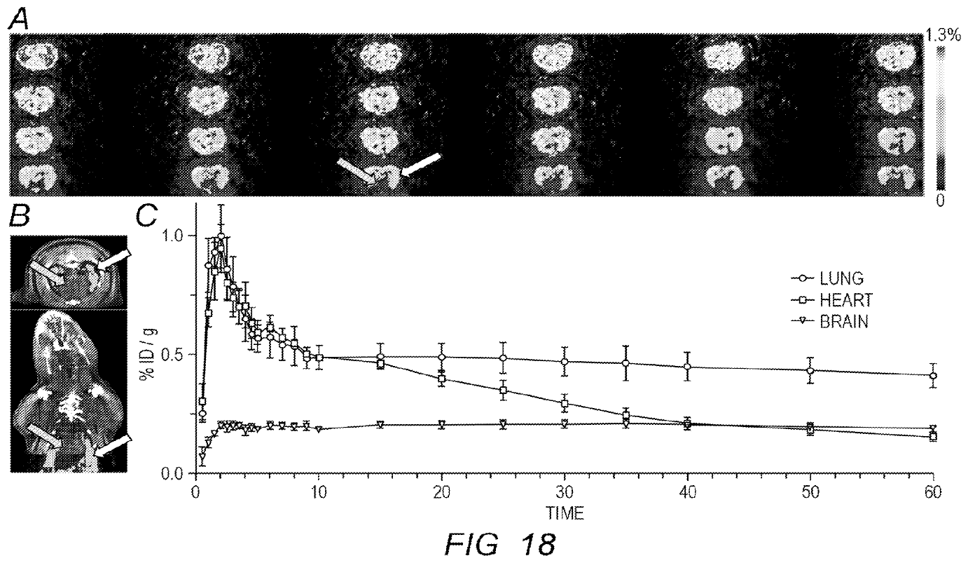

Similar to CSR positive gland, from the dynamic PET images and merged PET/CT images (FIG. 18 panel A and panel B), it can be seen that the lung exhibited a high level of radiotracer uptake. According to regional time-radioactivity curves (FIG. 18C), the radiotracer uptake in the lung showed a “fast in and fast out” trend. Uptake peaked at 2 min post injection (lung: 1.00±0.19% ID/g; heart: 0.95±0.14% ID/g), with similar kinetics until 10 min post injection. Then, the heart uptake dropped rapidly to 0.15±0.27% ID/g at 60 min post injection, whereas the lung uptake remained at a high level at 60 min post injection of 0.41±0.07% ID/g. In addition, the radiotracer was also observed entering into the brain with a stable uptake level of ˜0.2% ID/g.

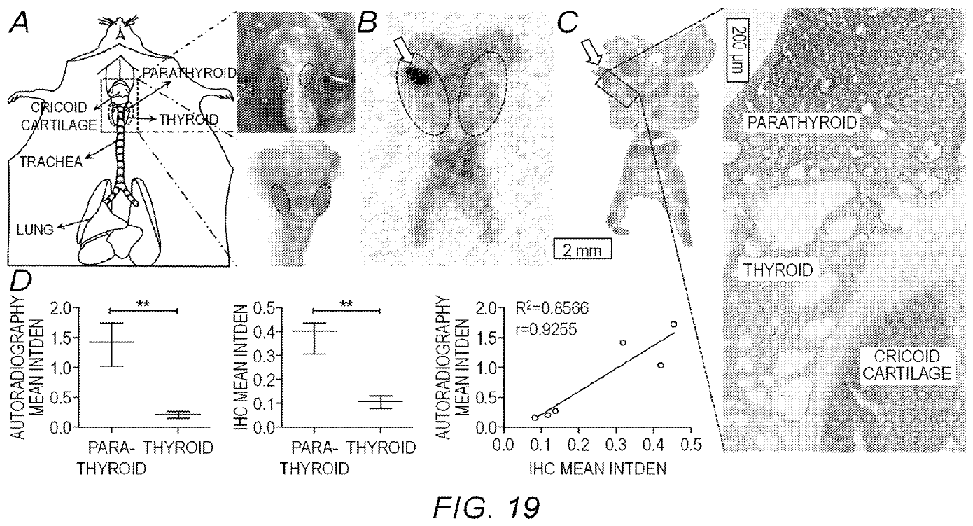

Autoradiography and pathology. To further confirm the imaging results of parathyroids from [¹⁸F]F-ZW-cinacalcet-based PET/CT, ex-vivo studies were conducted to verify the accuracy of radiotracer targeting by autoradiography and IHC staining. First, the regional larynx and tracheal tissues containing thyroid and parathyroid (FIG. 19 panel A) were excised from the rats injected with [¹⁸F]F-ZW-cinacalcet for immediate cutting sections, which were used for autoradiography and IHC staining of CSR, respectively. As shown in FIG. 19 panels B and C, autoradiography matched well with CSR IHC staining. The highest radioactive uptake area on autoradiography matched with the IHC-stained parathyroid area. Quantitative analysis of autoradiography and IHC staining was expressed as a mean integrated density (Mean IntDen). The uptake of the radiotracer and CSR expression in the parathyroid were significantly higher than those in the thyroid background tissues (P<0.01), with parathyroid/thyroid ratios of 6.60±1.28 in autoradiography and 3.76±1.13 in IHC staining (FIG. 19 panel D). Meanwhile, the CSR expression level correlated well with the radiotracer uptake (r=0.93, 95% confidence interval 0.46˜0.99, R²=0.86) in the parathyroid and thyroid (FIG. 19 panel D).

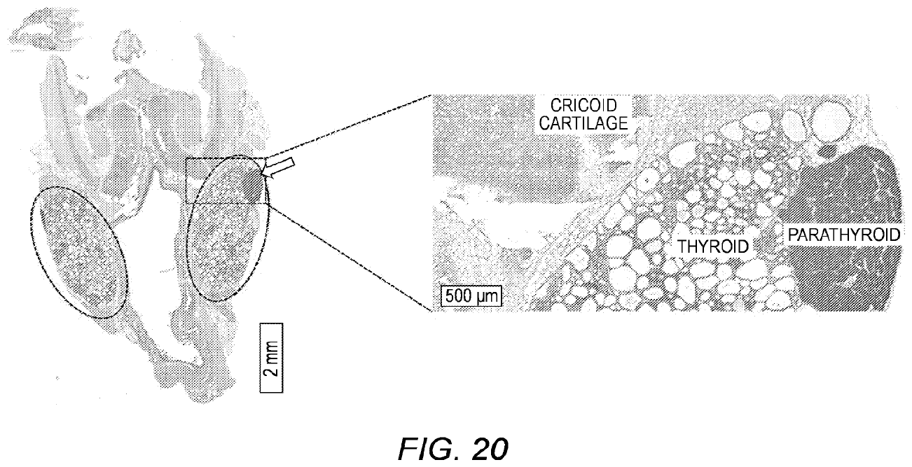

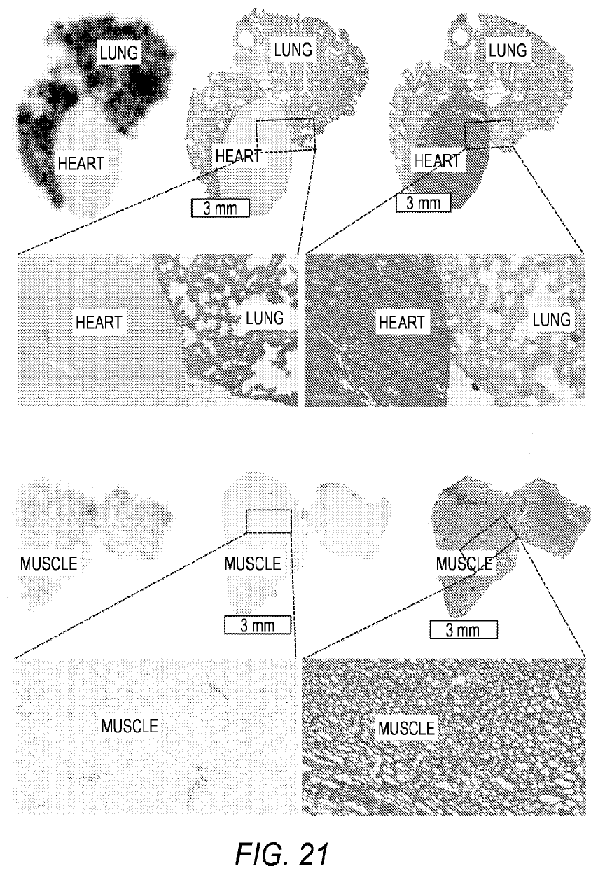

To better maintain structural integrity, some tissues were also fixed with formalin and embedded in paraffin to generate cutting sections for CSR-IHC staining. The highest expression level of CSR was observed in the parathyroid (FIG. 20), consistent with the IHC staining results using frozen tissues. The high uptake of radioactivity in the lung was observed in PET imaging. We also performed autoradiography and IHC staining of the lung (along with the heart) and muscles to verify the observations. The results showed that the lung had extremely high expression of CSR compared to the heart and muscle (FIG. 21), and matched well with the autoradiography data (FIG. 21), IHC and H&E staining. This explained the high uptake of the [¹⁸F]F-cinacalcet found in the lung using PET/CT imaging.

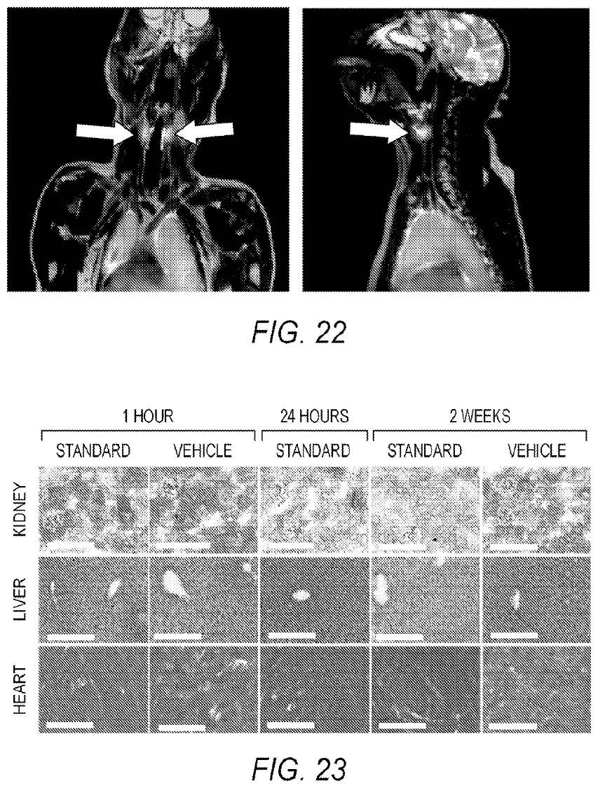

Clinical PET MRI imaging of parathyroid in nonhuman primates. To further facilitate future clinical translation, PET/MRI scan in non-human-primates (NHPs) was performed (FIG. 22). [¹⁸F]F-ZW-cinacalcet demonstrated apparent tissue accumulation in the parathyroid region.

In vivo Toxicity study. An In vivo acute toxicity study of [¹⁹F]F-cinacalcet was carried out in JAX Swiss Outbred mice. The mice were injected intravenously with an overdose of cold standard [¹⁹F]F-cinacalcet (57.6 μg, ˜1000 times greater than the radiotracer [¹⁸F]F-cinacalcet dose). Plasma samples were collected and analyzed at various time points. No animal deaths and significant health changes were observed during the study period. Alkaline phosphatase (ALP) and alanine transaminase (ALT) are two important biological indicators associated with liver function. Plasma test results showed that there were no significant changes in ALP and ALT levels in the treated mice (Table 1). A slight but insignificant increase in AST was observed at 1 hour after injection, reduced quickly within 24 hours, and returned to normal levels after 2 weeks. It is very common to see a transient increase in blood AST after drug administration, which usually has no actual pathological significance. Blood urea nitrogen (BUN) and creatinine are two critical indicators for evaluating kidney function. There were no significant changes in BUN and creatinine levels in the mice injected with [¹⁹F]F-cinacalcet compared with the vehicle controls. In addition, histopathological staining was performed on the kidney, liver, and heart tissues collected at different time points. As shown in FIG. 23, no obvious pathological changes were observed in the mice with [¹⁹F]F-cinacalcet administration. The basic structures of the glomerulus, renal tubulus, liver lobule central vein, and cardiac muscle fibers remained intact. All these results showed that overdosage of cold standard [¹⁹F]F-cinacalcet had no acute toxicity to mice, indicating the biological safety of the radioprobe [¹⁸F]F-cinacalcet was well within tolerance limits.

TABLE 1
Changes of key biological indicators associated with liver and kidney
function after overdose of [¹⁹F]F-cinacalcet at different time points.

| | 1 h | 1 h | 24 h | 2 weeks | 2 weeks |
|---|---|---|---|---|---|
| | standard | vehicle | standard | standard | vehicle |
| ALP | 87.67 ± 13.70 | 71.33 ± 6.85 | 54 ± 9.09 | 80.33 ± 9.00 | 78.67 ± 4.99 |
| ALT | 29.33 ± 3.10 | 48.33 ± 3.86 | 31.33 ± 9.10 | 24.67 ± 0.94 | 29 ± 4.32 |
| AST | 82.67 ± 21.68 | 186.33 ± 77.56 | 104.67 ± 45.38 | 82 ± 28.6 | 90.67 ± 12.25 |
| BUN | 17 ± 1.41 | 21 ± 2.16 | 18.33 ± 1.25 | 18 ± 0.82 | 22 ± 0.82 |
| creatinine | 0.24 ± 0.09 | 0.24 ± 0.08 | 0.25 ± 0.11 | 0.18 ± 0.01 | 0.17 ± 0.03 |

ALP—alkaline phosphatase (U/L), ALT—alanine aminotransferase (U/L), AST—aminotransferase (U/L), BUN—blood urea nitrogen (mg/dL), creatinine (mg/dL).

In summary, acting as a calcimimetic, cinacalcet is a commonly used medication to treat PHPT. Considering the high expression of CSR in parathyroid cells, ¹⁸F labeled cinacalcet was hypothesized to act as a PET agent for parathyroid detection. Although previous attempts have been made to generate PET agents based on cinacalcet, the fast metabolism of previously labeled agents may be the main reason for the suboptimal results. In fact, the availability of novel PET agents may be limited due to the lack of efficient and simple labeling methods to modify biologically active small molecules/drugs. Herein, a highly innovative photoredox systems weas used, which allowed direct conversion of cinacalcet to a PET agent through arene C—H fluorination.

1. A radioisotope-labeled calcium sensing receptor (CSR) ligand comprising an aromatic ring, wherein the radioisotope is directly linked to the aromatic ring at one or more positions of the ring.

2. The radioisotope-labeled CSR ligand of claim 1, wherein the radioisotope is ¹⁸F and/or ¹¹C.

3-10. (canceled)

11. The radioisotope-labeled CSR ligand of claim 1, comprising formula II: 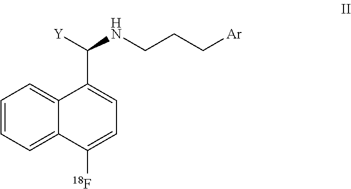
wherein Ar is substituted aryl and Y is alkyl.

12. (canceled)

13. The radioisotope-labeled CSR ligand of claim 11, comprising Formula III:

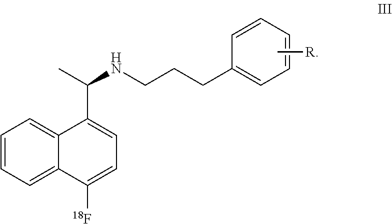

14. The radioisotope-labeled CSR ligand of claim 1, selected from the group consisting of:

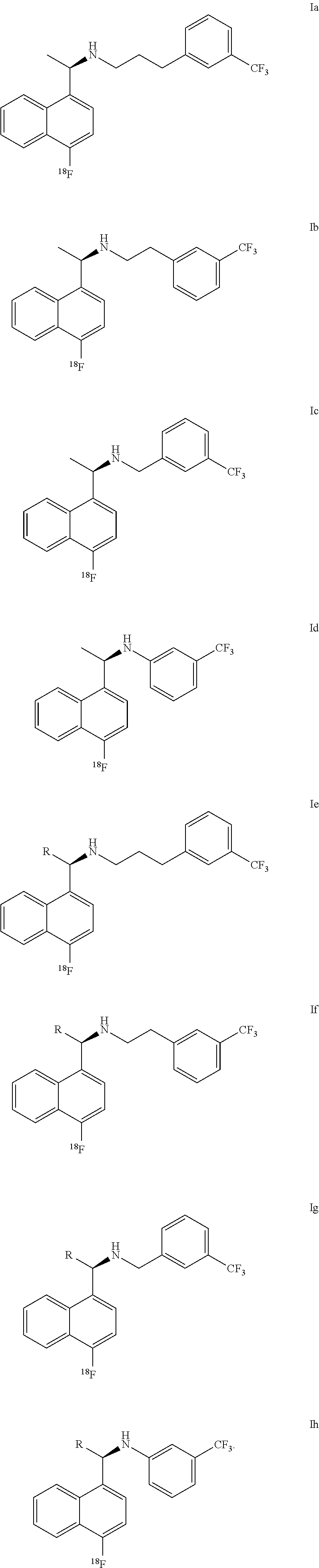

15. The radioisotope-labeled CSR ligand of claim 13, comprising formula I (¹⁸F-cinacalcet):

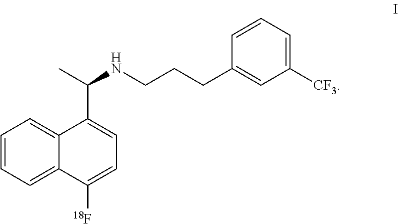 [SMILES: C[C@@H](NCCCc1cccc(C(F)(F)F)c1)c1ccc([18F])c2ccccc12]

16. The radioisotope-labeled CSR ligand of claim 1, comprising a formula selected from the group consisting of:

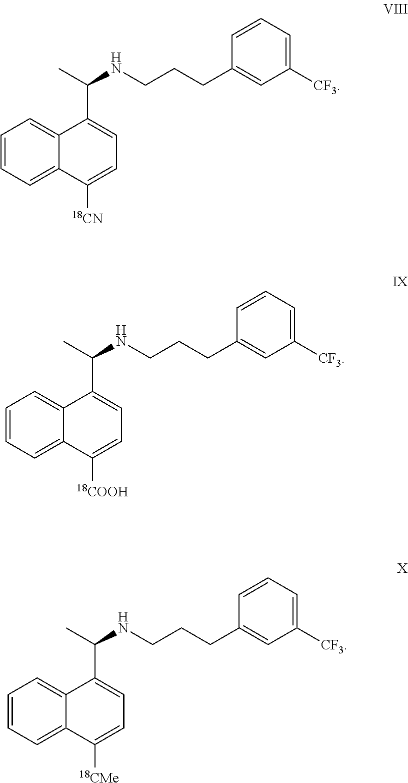

17. The radioisotope-labeled CSR ligand of claim 1, comprising a formula selected from the group consisting of:

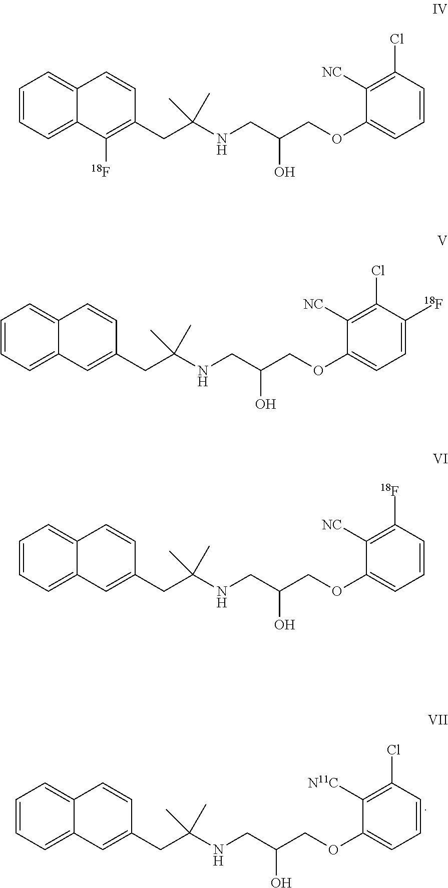

18. The radioisotope-labeled CSR ligand of claim 1, comprising formula XI (evocalcet): 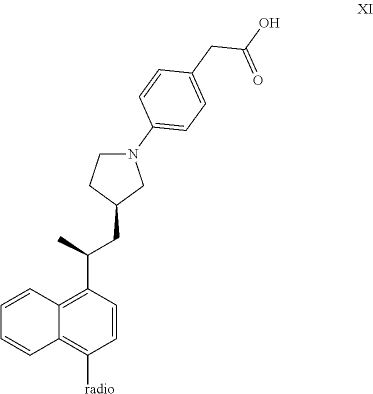
wherein radioisotope is directly linked to the naphthalene at position 4.

19. The radioisotope-labeled CSR ligand of claim 18, selected from the group consisting of:

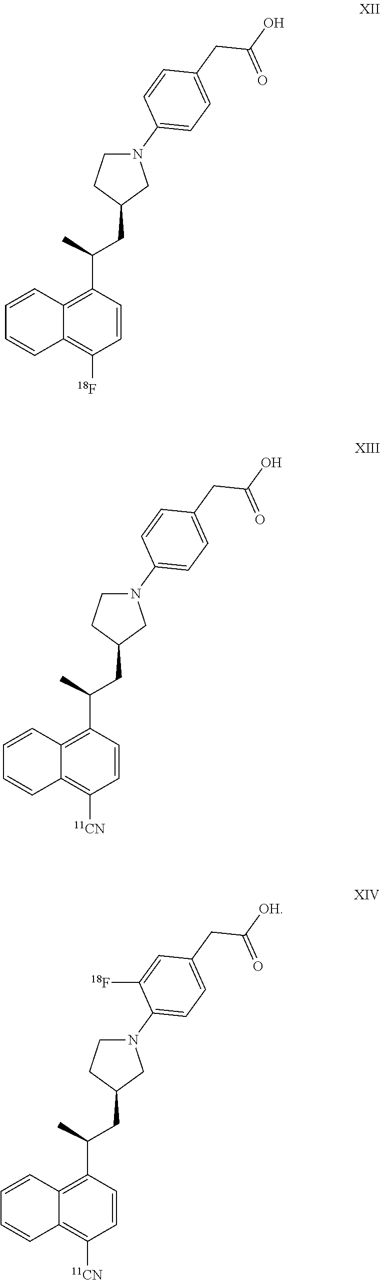

20. The radioisotope-labeled CSR ligand of claim 1, selected from the group consisting of:

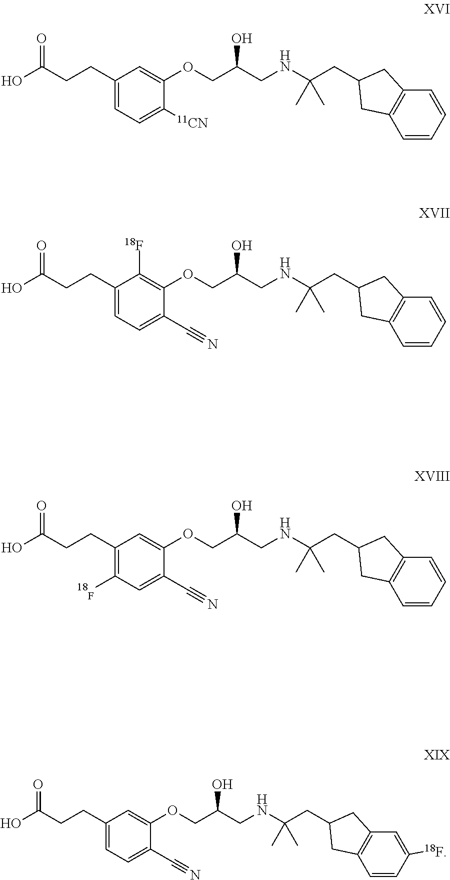

21. A radioisotope-labeled calcium sensing receptor (CSR) ligand comprising formula XX (etelcalcetide hydrochloride) directly linked to ¹⁸F:

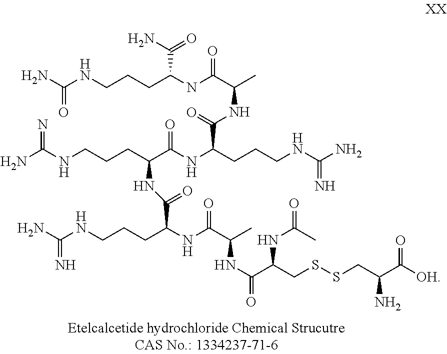

22. A labeled calcium sensing receptor (CSR) ligand suitable for use as a positron emission tomography (PET), fluorescence imaging (e.g., NIRF) probe and/or optical probe, comprising an CSR binding portion.

23-27. (canceled)

28. A halogenated fluorophore comprising a radioisotope, capable of preferential uptake in thyroid and/or parathyroid tissue.

29. (canceled)

30. The halogenated fluorophore of claim 28, comprising formula XXII
(¹⁸F-T700):

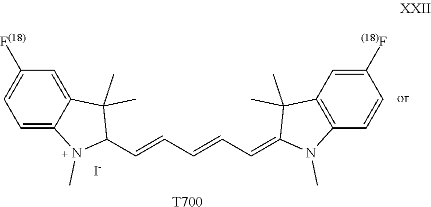

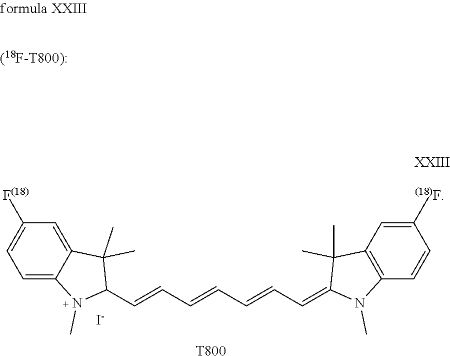

31-33. (canceled)

34. A positron emission tomography (PET) probe comprising the ligand or fluorophore of claim 1.

35. A fluorescence imaging probe (e.g., NIRF probe) comprising the ligand or fluorophore of claim 1.

36-40. (canceled)

41. A method of carrying out a PET scan on a subject, comprising administering to the subject the ligand, fluorophore, probe, and/or composition of claim 1.

42. A method of imaging tissue comprising a calcium sensing receptor (CSR) in a subject, comprising administering to the subject the ligand, fluorophore, probe, and/or composition of claim 1.

43. A method of imaging thyroid and/or parathyroid tissue in a subject, comprising administering to the subject the ligand, fluorophore, probe, and/or composition of claim 1.

44. A method of simultaneously carrying out a PET scan and fluorescence imaging (e.g., NIRF imaging) on a subject, comprising administering to the subject the ligand, fluorophore, probe, and/or composition of claim 1.

45. A method of identifying parathyroid tissue in a subject, comprising carrying out a PET scan and/or fluorescence imaging (e.g., NIRF imaging) on the subject using the ligand, fluorophore, probe, or composition of claim 1, wherein the PET scan and/or fluorescence imaging identifies presence of parathyroid tissue.

46-57. (canceled)

58. A method of removing abnormal (e.g., adenomatous, hyperplastic, malignant, and/or ectopic parathyroid) tissue in a subject, comprising:
(a) carrying out a PET scan and/or fluorescence imaging (e.g., NIRF imaging) on the subject using the ligand, fluorophore, probe, and/or composition of claim 1, wherein the PET scan and/or fluorescence imaging identifies presence of parathyroid tissue;
(b) identifying present parathyroid tissue that is abnormal (e.g., adenomatous, hyperplastic, malignant, and/or ectopic); and
(C) surgically excising the identified abnormal (e.g., adenomatous, hyperplastic, malignant, and/or ectopic) parathyroid tissue, thereby removing the abnormal (e.g., adenomatous, hyperplastic, malignant, and/or ectopic) parathyroid tissue.

59. (canceled)

60. A method of guiding surgery for the removal of parathyroid tissue in a subject, comprising carrying out a PET scan and/or fluorescence imaging (e.g., NIRF imaging) on the subject using the ligand, fluorophore, probe, and/or composition of claim 1, wherein the PET scan and/or fluorescence imaging identifies presence of parathyroid tissue.

61-64. (canceled)

65. A method of guiding surgery for the protection of parathyroid tissue during thyroid surgery in a subject, comprising carrying out a PET scan and/or fluorescence imaging (e.g., NIRF imaging) on the subject using the ligand, fluorophore, probe, and/or composition of claim 1, wherein the PET scan and/or fluorescence imaging identifies presence of parathyroid tissue.

66-70. (canceled)

71. A method of determining target regions for surgical removal of parathyroid tissue of a subject with hyperparathyroidism or a subject at risk for or suspected to have or develop hyperparathyroidism, comprising
(a) carrying out a PET scan and/or fluorescence imaging (e.g., NIRF imaging) on the subject using the ligand, fluorophore, probe, and/or composition of claim 1, wherein the PET scan and/or fluorescence imaging identifies presence of parathyroid tissue; and
(b) identifying one or more regions(s) of the subject comprising the presence of adenomatous, hyperplastic, malignant, and/or ectopic parathyroid tissue, wherein the presence of parathyroid tissue in the one or more region(s) indicates the region(s) as a target region of the subject for surgical removal of parathyroid tissue.

72-76. (canceled)

77. A method of treating hyperparathyroidism in a subject, comprising determining the suitability of a subject with hyperparathyroidism or a subject at risk for or suspected to have or develop hyperparathyroidism to surgical removal of parathyroid tissue by carrying out a PET scan on the subject using the ligand, fluorophore, probe, or composition of claim 1, wherein the PET scan identifies presence of parathyroid tissue, and treating the hyperparathyroidism based on the results of the PET scan.

78-80. (canceled)

81. A method of treating a disorder of a calcium sensing receptor (CSR)-positive tissue in a subject, comprising determining suitability of a subject with the disorder or a subject at risk for or suspected to have or develop the disorder to treatment thereof by carrying out a PET scan on the subject using the ligand, fluorophore, probe, or composition of claim 1, wherein the PET scan identifies presence of CSR-positive tissue, and treating the disorder based on the results of the PET scan.

82-87. (canceled)
